SEQUENCE LISTING (111 sequences)

SEQ ID NO: 1 - DNA, 605 bp, Artificial Sequence
  Description of Artificial Sequence first and second exons of human heparanase gene
cagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctggggtaagcgc

SEQ ID NO: 2 - DNA, 813 bp, Artificial Sequence
  Description of Artificial Sequence new human heparanase upstream sequence
gtgtcatggagagctgcctggagattgagagaaagcttccttgagggaagttacatttcagctgaaacacactgccatctgctcgaggttttgtaactgcattcacatcccgattctgacacttcacatcccgattctgacacttcacccagttactgtctcagagcttgggtccgcatgtgtaaaacaaggacagtatgcacttggcagggttgtgagaagggaagagaacacaagtaaagcacctgtatcaggcatacagtaggcactaagcgtgcgatgcttgctatgattatacatcagtgtaagcagcaaggaaaagctgaagaaaagtctgaccaacagcgaaagataaatgcgcagaggagaaatttggcaaaggctccaaattcaggggcagtccgtactctacactttgtatgggggcttcaggtcctgagttccagacattggagcaactaaccctttaagattgctaaatattgtcttaatgagaagttgataaagaattttgggtggttgatctctttccagctgcagtttagcgtatgctgaggccagattttttcaagcaaaagtaaaatacctgagaaactgcctggccagaggacaatcagattttggctggctcaagtgacaagcaagtgtttataagctagatgggaggggaagggatgaatactccattggaggttttactcgagggtcagagggatacccggcgccatcagaatgggatctgggagtcggaaacgctgggttcccacgagagcgcgcagaacacgtgcgtcaggaagcctggtccgggatgcc

SEQ ID NO: 3 - DNA, 746 bp, Artificial Sequence
  Description of Artificial Sequence new human heparanase downstream sequence
cagcctcctggtcctgtcccctttcctgtcctcctgacacctatgtctgccccgccagccgctctccttcttttgcgcggaaacaacttcacaccggaacctccccgcctgtctctccccaccccacttcccgcctctcattctccctctccctcccttactctcagaccccaaaccgctttttggggggtatcatttaaaaaatagatttaggggttacaagtgcagttctgttccatgggtatattgcattgtggtggcatctgggctcttagtgtaactgtcacccgaatgttgtacattgtatctaataggtaatttctcatcctcatccctctcccaccctcccaccttttggagtctccagtgtctactattccactaagtccatgtgtacacattgtttagcgcccactctaaatgagcctttttgtttcattcattctgtaagtgttgaataggcaccacctaaggtcaggtataagtggaaatttgaaaaggaaactgcccacttgccccagtacttccctagccaagaggagggaaaccaggcaggtgcacctgaaggcctgtgagtgcttgatttgctgtgcagtgtaggacaagtaagattgtgcatagccttctgtatttaagactgtgttaggaagatttctctttcttttcttttctttttcttttttcttttcttttttttttttaggcagatgaaaagggcgtcacagaacaggaataaaaatctaaat

SEQ ID NO: 4 - DNA, 1419 bp, Artificial Sequence
  Description of Artificial Sequence human heparanase gene in the vicinity of the upstream region
gtgtcatggagagctgcctggagattgagagaaagcttccttgagggaagttacatttcagctgaaacacactgccatctgctcgaggttttgtaactgcattcacatcccgattctgacacttcacatcccgattctgacacttcacccagttactgtctcagagcttgggtccgcatgtgtaaaacaaggacagtatgcacttggcagggttgtgagaagggaagagaacacaagtaaagcacctgtatcaggcatacagtaggcactaagcgtgcgatgcttgctatgattatacatcagtgtaagcagcaaggaaaagctgaagaaaagtctgaccaacagcgaaagataaatgcgcagaggagaaatttggcaaaggctccaaattcaggggcagtccgtactctacacttttgtatgggggcttcaggtcctgagttccagacattggagcaactaaccctttaagattgctaaatattgtcttaatgagaagttgataaagaattttgggtggttgatctctttccagctgcagtttagcgtatgctgaggccagattttttcaagcaaaagtaaaatacctgagaaactgcctggccagaggacaatcagattttggctggctcaagtgacaagcaagtgtttataagctagatgggaggggaagggatgaatactccattggaggttttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctggggtaagcgc

SEQ ID NO: 5 - DNA, 30 bp, Artificial Sequence
  Description of Artificial Sequence primer HP-5
catatgtccgtcaccattgacgccaacctg

SEQ ID NO: 6 - DNA, 30 bp, Artificial Sequence
  Description of Artificial Sequence primer HP-6
ctgcaggacgtcgtggacctggacttcttc

SEQ ID NO: 7 - DNA, 28 bp, Artificial Sequence
  Description of Artificial Sequence primer HP-7
tcgcgaaatcacccacacccacttgaag

SEQ ID NO: 8 - DNA, 26 bp, Artificial Sequence
  Description of Artificial Sequence primer HP-8
gccggctctctcctacttccttgctc

SEQ ID NO: 9 - DNA, 9 bp, Artificial Sequence
  Description of Artificial Sequence SP1 binding site
ggggcgggg

SEQ ID NO: 10 - DNA, 6 bp, Artificial Sequence
  Description of Artificial Sequence EST1 binding site
aggaag

SEQ ID NO: 11 - DNA, 6 bp, Artificial Sequence
  Description of Artificial Sequence AP1 binding site
gcgtca

SEQ ID NO: 12 - DNA, 6 bp, Artificial Sequence
  Description of Artificial Sequence E box sequence
casswg

SEQ ID NO: 13 - DNA, 6 bp, Artificial Sequence
  Description of Artificial Sequence IK-2 binding site
tgggad

SEQ ID NO: 14 - DNA, 20 bp, Artificial Sequence
  Description of Artificial Sequence forward strand
caagcacaggacgtcgtgga

SEQ ID NO: 15 - DNA, 25 bp, Artificial Sequence
  Description of Artificial Sequence probe
ctcgttcctgtccgtcaccattgac

SEQ ID NO: 16 - DNA, 20 bp, Artificial Sequence
  Description of Artificial Sequence reverse strand
agcctctggccaaggtacga

SEQ ID NO: 17 - DNA, 500 bp, Artificial Sequence
  Description of Artificial Sequence region of the human heparanase gene located upstream of the translation initiation site
tcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcga

SEQ ID NO: 18 - DNA, 404 bp, Artificial Sequence
  Description of Artificial Sequence human heparanase gene regulatory region
ggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgcta

SEQ ID NO: 19 - DNA, 9 bp, Artificial Sequence
  Description of Artificial Sequence SBS# 781 target
ggggagcag

SEQ ID NO: 20 - DNA, 9 bp, Artificial Sequence
  Description of Artificial Sequence SBS# 779 target
cggggagca

SEQ ID NO: 21 - DNA, 9 bp, Artificial Sequence
  Description of Artificial Sequence SBS# 519 target
ggggaggag

SEQ ID NO: 22 - DNA, 9 bp, Artificial Sequence
  Description of Artificial Sequence SBS# 1756 target
cggggagga

SEQ ID NO: 23 - DNA, 9 bp, Artificial Sequence
  Description of Artificial Sequence SBS# 519 target
ggggaggag

SEQ ID NO: 24 - DNA, 9 bp, Artificial Sequence
  Description of Artificial Sequence SBS# 1755 target
cgggaggcc

SEQ ID NO: 25 - DNA, 9 bp, Artificial Sequence
  Description of Artificial Sequence SBS# 1760 target
atggccggg

SEQ ID NO: 26 - DNA, 9 bp, Artificial Sequence
  Description of Artificial Sequence SBS# 1757 target
ggtgcggag

SEQ ID NO: 27 - DNA, 9 bp, Artificial Sequence
  Description of Artificial Sequence SBS# 1140 target
aagggtgcg

SEQ ID NO: 28 - DNA, 9 bp, Artificial Sequence
  Description of Artificial Sequence SBS# 1764 target
caagtgggt

SEQ ID NO: 29 - DNA, 9 bp, Artificial Sequence
  Description of Artificial Sequence SBS# 1772 target
gtgggtgat

SEQ ID NO: 30 - DNA, 9 bp, Artificial Sequence
  Description of Artificial Sequence SBS# 705 target
ggggcgggg

SEQ ID NO: 31 - DNA, 9 bp, Artificial Sequence
  Description of Artificial Sequence SBS# 1750 target
atggagggc

SEQ ID NO: 32 - DNA, 9 bp, Artificial Sequence
  Description of Artificial Sequence SBS# 713 target
ggtgaggag

SEQ ID NO: 33 - DNA, 9 bp, Artificial Sequence
  Description of Artificial Sequence SBS# 1796 target
gaggaggcg

SEQ ID NO: 34 - DNA, 9 bp, Artificial Sequence
  Description of Artificial Sequence SBS# 386 target
ggggcggag

SEQ ID NO: 35 - protein, 7 aa, Artificial Sequence
  Description of Artificial Sequence SBS# 781 Finger1
RSSNLRE

SEQ ID NO: 36 - protein, 7 aa, Artificial Sequence
  Description of Artificial Sequence SBS# 781 Finger2
RSDNLAR

SEQ ID NO: 37 - protein, 7 aa, Artificial Sequence
  Description of Artificial Sequence SBS# 781 Finger3
RSDHLTR

SEQ ID NO: 38 - protein, 7 aa, Artificial Sequence
  Description of Artificial Sequence SBS# 779 Finger1
QSGSLTR

SEQ ID NO: 39 - protein, 7 aa, Artificial Sequence
  Description of Artificial Sequence SBS# 779 Finger2
QSGHLTR

SEQ ID NO: 40 - protein, 7 aa, Artificial Sequence
  Description of Artificial Sequence SBS# 779 Finger3
RSDHLAE

SEQ ID NO: 41 - protein, 7 aa, Artificial Sequence
  Description of Artificial Sequence SBS# 519 Finger1
RSDNLTR

SEQ ID NO: 42 - protein, 7 aa, Artificial Sequence
  Description of Artificial Sequence SBS# 519 Finger2
RSDNLAR

SEQ ID NO: 43 - protein, 7 aa, Artificial Sequence
  Description of Artificial Sequence SBS# 519 Finger3
RSDHLSR

SEQ ID NO: 44 - protein, 7 aa, Artificial Sequence
  Description of Artificial Sequence SBS# 1756 Finger1
QQAHLAR

SEQ ID NO: 45 - protein, 7 aa, Artificial Sequence
  Description of Artificial Sequence SBS# 1756 Finger2
QSGHLQR

SEQ ID NO: 46 - protein, 7 aa, Artificial Sequence
  Description of Artificial Sequence SBS# 1756 Finger3
RSDHLRE

SEQ ID NO: 47 - protein, 7 aa, Artificial Sequence
  Description of Artificial Sequence SBS# 519 Finger1
RSDNLTR

SEQ ID NO: 48 - protein, 7 aa, Artificial Sequence
  Description of Artificial Sequence SBS# 519 Finger2
RSDNLAR

SEQ ID NO: 49 - protein, 7 aa, Artificial Sequence
  Description of Artificial Sequence SBS# 519 Finger3
RSDHLSR

SEQ ID NO: 50 - protein, 7 aa, Artificial Sequence
  Description of Artificial Sequence SBS# 1755 Finger1
ERGTLAR

SEQ ID NO: 51 - protein, 7 aa, Artificial Sequence
  Description of Artificial Sequence SBS# 1755 Finger2
RSDNLAR

SEQ ID NO: 52 - protein, 7 aa, Artificial Sequence
  Description of Artificial Sequence SBS# 1755 Finger3
RSDHLRE

SEQ ID NO: 53 - protein, 7 aa, Artificial Sequence
  Description of Artificial Sequence SBS# 1760 Finger1
RSDHLAR

SEQ ID NO: 54 - protein, 7 aa, Artificial Sequence
  Description of Artificial Sequence SBS# 1760 Finger2
DCRDLAR

SEQ ID NO: 55 - protein, 7 aa, Artificial Sequence
  Description of Artificial Sequence SBS# 1760 Finger3
RSDALTQ

SEQ ID NO: 56 - protein, 7 aa, Artificial Sequence
  Description of Artificial Sequence SBS# 1757 Finger1
RSDNLTR

SEQ ID NO: 57 - protein, 7 aa, Artificial Sequence
  Description of Artificial Sequence SBS# 1757 Finger2
RSDDLNR

SEQ ID NO: 58 - protein, 7 aa, Artificial Sequence
  Description of Artificial Sequence SBS# 1757 Finger3
TSGHLVR

SEQ ID NO: 59 - protein, 7 aa, Artificial Sequence
  Description of Artificial Sequence SBS# 1140 Finger1
RSDELTR

SEQ ID NO: 60 - protein, 7 aa, Artificial Sequence
  Description of Artificial Sequence SBS# 1140 Finger2
QSSHLAR

SEQ ID NO: 61 - protein, 7 aa, Artificial Sequence
  Description of Artificial Sequence SBS# 1140 Finger3
RSDNLTQ

SEQ ID NO: 62 - protein, 7 aa, Artificial Sequence
  Description of Artificial Sequence SBS# 1764 Finger1
QSGHLAR

SEQ ID NO: 63 - protein, 7 aa, Artificial Sequence
  Description of Artificial Sequence SBS# 1764 Finger2
RSDALAR

SEQ ID NO: 64 - protein, 7 aa, Artificial Sequence
  Description of Artificial Sequence SBS# 1764 Finger3
QSGNLTE

SEQ ID NO: 65 - protein, 7 aa, Artificial Sequence
  Description of Artificial Sequence SBS# 1772 Finger1
TTSNLAR

SEQ ID NO: 66 - protein, 7 aa, Artificial Sequence
  Description of Artificial Sequence SBS# 1772 Finger2
QSSHLAR

SEQ ID NO: 67 - protein, 7 aa, Artificial Sequence
  Description of Artificial Sequence SBS# 1772 Finger3
RSDALTR

SEQ ID NO: 68 - protein, 7 aa, Artificial Sequence
  Description of Artificial Sequence SBS# 705 Finger1
RSDHLAR

SEQ ID NO: 69 - protein, 7 aa, Artificial Sequence
  Description of Artificial Sequence SBS# 705 Finger2
RSDSLAR

SEQ ID NO: 70 - protein, 7 aa, Artificial Sequence
  Description of Artificial Sequence SBS# 705 Finger3
RSDHLSR

SEQ ID NO: 71 - protein, 7 aa, Artificial Sequence
  Description of Artificial Sequence SBS# 1750 Finger1
DRSHLTR

SEQ ID NO: 72 - protein, 7 aa, Artificial Sequence
  Description of Artificial Sequence SBS# 1750 Finger2
RSDNLAR

SEQ ID NO: 73 - protein, 7 aa, Artificial Sequence
  Description of Artificial Sequence SBS# 1750 Finger3
RSDALTE

SEQ ID NO: 74 - protein, 7 aa, Artificial Sequence
  Description of Artificial Sequence SBS# 713 Finger1
RSDNLAR

SEQ ID NO: 75 - protein, 7 aa, Artificial Sequence
  Description of Artificial Sequence SBS# 713 Finger2
RSDNLAR

SEQ ID NO: 76 - protein, 7 aa, Artificial Sequence
  Description of Artificial Sequence SBS# 713 Finger3
MSHHLSR

SEQ ID NO: 77 - protein, 7 aa, Artificial Sequence
  Description of Artificial Sequence SBS# 1796 Finger1
RSDDLTR

SEQ ID NO: 78 - protein, 7 aa, Artificial Sequence
  Description of Artificial Sequence SBS# 1796 Finger2
RSDNLAR

SEQ ID NO: 79 - protein, 7 aa, Artificial Sequence
  Description of Artificial Sequence SBS# 1796 Finger3
RSDNLAR

SEQ ID NO: 80 - protein, 7 aa, Artificial Sequence
  Description of Artificial Sequence SBS# 386 Finger1
RSDNLTR

SEQ ID NO: 81 - protein, 7 aa, Artificial Sequence
  Description of Artificial Sequence SBS# 386 Finger2
RSDELQR

SEQ ID NO: 82 - protein, 7 aa, Artificial Sequence
  Description of Artificial Sequence SBS# 386 Finger3
RSDHLSR

SEQ ID NO: 83 - protein, 7 aa, Artificial Sequence
  Description of Artificial Sequence SBS# 1610 Finger1
QSSDLRR

SEQ ID NO: 84 - protein, 7 aa, Artificial Sequence
  Description of Artificial Sequence SBS# 1610 Finger2
RSDHLTR

SEQ ID NO: 85 - protein, 7 aa, Artificial Sequence
  Description of Artificial Sequence SBS# 1610 Finger3
QSSDLRR

SEQ ID NO: 86 - protein, 7 aa, Artificial Sequence
  Description of Artificial Sequence SBS# 1770 Finger1
QRAHLER

SEQ ID NO: 87 - protein, 7 aa, Artificial Sequence
  Description of Artificial Sequence SBS# 1770 Finger2
QSGHLQR

SEQ ID NO: 88 - protein, 7 aa, Artificial Sequence
  Description of Artificial Sequence SBS# 1770 Finger3
RSDHLRE

SEQ ID NO: 89 - DNA, 18 bp, Artificial Sequence
  Description of Artificial Sequence SBS# 5348 target
ggtgcggagatggccggg

SEQ ID NO: 90 - DNA, 19 bp, Artificial Sequence
  Description of Artificial Sequence SBS# 5349 target
cgggaggccaggggaggag

SEQ ID NO: 91 - DNA, 18 bp, Artificial Sequence
  Description of Artificial Sequence SBS# 5468 target
caagtgggtgtgggtgat

SEQ ID NO: 92 - protein, 7 aa, Artificial Sequence
  Description of Artificial Sequence SBS# 5348 F1
RSAHLAR

SEQ ID NO: 93 - protein, 7 aa, Artificial Sequence
  Description of Artificial Sequence SBS# 5348 F2
DRSDLAR

SEQ ID NO: 94 - protein, 7 aa, Artificial Sequence
  Description of Artificial Sequence SBS# 5348 F3
RSDALTQ

SEQ ID NO: 95 - protein, 7 aa, Artificial Sequence
  Description of Artificial Sequence SBS# 5348 F4
RSANLAR

SEQ ID NO: 96 - protein, 7 aa, Artificial Sequence
  Description of Artificial Sequence SBS# 5348 F5
RSDDLNR

SEQ ID NO: 97 - protein, 7 aa, Artificial Sequence
  Description of Artificial Sequence SBS# 5348 F6
TSGHLVR

SEQ ID NO: 98 - protein, 7 aa, Artificial Sequence
  Description of Artificial Sequence SBS# 5349 F1
RSDNLTR

SEQ ID NO: 99 - protein, 7 aa, Artificial Sequence
  Description of Artificial Sequence SBS# 5349 F2
RSDNLAR

SEQ ID NO: 100 - protein, 7 aa, Artificial Sequence
  Description of Artificial Sequence SBS# 5349 F3
RSDHLSR

SEQ ID NO: 101 - protein, 7 aa, Artificial Sequence
  Description of Artificial Sequence SBS# 5349 F4
ERGTLAR

SEQ ID NO: 102 - protein, 7 aa, Artificial Sequence
  Description of Artificial Sequence SBS# 5349 F5
RSDNALR

SEQ ID NO: 103 - protein, 7 aa, Artificial Sequence
  Description of Artificial Sequence SBS# 5349 F6
RSDHLRE

SEQ ID NO: 104 - protein, 7 aa, Artificial Sequence
  Description of Artificial Sequence SBS# 5468 F1
TTSNLAR

SEQ ID NO: 105 - protein, 7 aa, Artificial Sequence
  Description of Artificial Sequence SBS# 5468 F2
QSSHLAR

SEQ ID NO: 106 - protein, 7 aa, Artificial Sequence
  Description of Artificial Sequence SBS# 5468 F3
RSDALTR

SEQ ID NO: 107 - protein, 7 aa, Artificial Sequence
  Description of Artificial Sequence SBS# 5468 F4
QSGHLAR

SEQ ID NO: 108 - protein, 7 aa, Artificial Sequence
  Description of Artificial Sequence SBS# 5468 F5
RSDALAR

SEQ ID NO: 109 - protein, 7 aa, Artificial Sequence
  Description of Artificial Sequence SBS# 5468 F6
QSTNLKS

SEQ ID NO: 110 - DNA, 9 bp, Artificial Sequence
  Description of Artificial Sequence SBS# 1610 target
gctggggct

SEQ ID NO: 111 - DNA, 9 bp, Artificial Sequence
  Description of Artificial Sequence SBS# 1770 target
cggggagga

(19) United States
(12) Patent Application Publication
(10) Pub. No.: US 2004/0132033 A1
(43) Pub. Date: Jul. 8, 2004

(54) Human heparanase gene regulatory sequences

(76) Inventors: Elizabeth J. Wolffe, Orinda, CA (US); Alan P. Wolffe, Orinda, CA (US); Hong Qi, Cottonwood, CA (US)

Correspondence Address: ROBINS & PASTERNAK, 1731 EMBARCADERO ROAD, SUITE 230, PALO ALTO, CA, 94303, US

(21) Appl. No.: 10/433,258

(22) PCT Filed: Nov. 30, 2001
(86) PCT No.: PCT/US01/44798

(51) Int. Cl. C12Q 1/68; C08B 37/10; C07H 21/04; C12N 9/24

(52) U.S. Cl. 435/6; 435/69.1; 435/200; 435/320.1; 435/325; 536/23.2; 536/21

(57) ABSTRACT

Nucleotide sequences comprising regulatory regions of the human heparanase gene are provided. Also provided are methods and compositions for regulating heparanase expression, as well as methods and compositions for using heparanase sequences to regulate a heterologous target gene.

### STATEMENT REGARDING FEDERALLY SPONSORED RESEARCH

Certain of the research described in this application was made with financial support from the United States government, in the form of grant No: 1R43CA86553-01, from the National Cancer Institute. Accordingly, the U.S. government may have certain rights to the claimed subject matter.

### TECHNICAL FIELD

This disclosure is in the field of molecular biology and medicine. More specifically, it relates to novel heparanase gene nucleotide sequences, and compositions and methods for modulating gene expression.

### BACKGROUND

Heparanase is an endoglycosidase that degrades the heparan sulfate proteoglycan of the extracellular matrix by invading cells, notably metastatic tumor cells and migrating leukocytes. For a description of heparanase, see, for example U.S. Pat. Nos. 5,362,641 and 5,968,822, incorporated by reference in their entireties herein. Transfection of rodent tumor cells with the heparanase gene enhances the metastatic potential of cells, providing direct evidence for a role of heparanase in invasion. Heparanase inhibitors (mainly based on heparin and similar polysaccharides) have been shown to inhibit tumor growth or metastasis, angiogenesis and vascular damage in some cases in experimental models.

Tumor metastasis requires neoangiogenesis and invasion of the basement membrane and extracellular matrix, which are largely composed of structural proteins and glycosaminoglycans, mainly heparan sulfate proteoglycans (HSPGs). Considerable attention has been focused on serine and cysteine proteases and matrix metalloproteinases (MMPs). These enzymes are often up regulated in metastatic cancers and proliferating endothelial cells (Eccles, S. A. (1999) Nat Med 5:735-6). Their substrates include collagens, laminin, fibronectin, and vitronectin. These protease activities not only enable tumor cells to break down tissue barriers and invade through stroma and blood vessel walls at primary and secondary sites, but also stimulate angiogenesis.

In addition to the proteases, cancer cells also produce heparanase that degrades the heparan sulfate side chain of HSPGs. This enzyme is normally found mainly in platelets, placental trophoblasts and leukocytes, and functions in embryonic morphogenesis, wound healing, tissue repair and inflammation (Ishai-Michaeli et al. (1990) Cell Regul 1:833-42). Heparanase released from activated platelets in response to vascular damage enables extravasation of inflammatory cells and stimulates endothelial mitogenesis. It not only assists in the breakdown of the extracellular matrix and the basement membrane, but also is involved in the regulation of growth factor and cytokine activity (Rapraeger et al. (1991) ^{Science} 252:1705-8). Tumor cells appear to use this same molecular machinery during metastasis and neoangiogenesis.

In contrast to our understanding of matrix metalloproteinases, the first cDNA sequence of mammalian heparanase has only just recently been reported (Vlodavsky et al. (1999) Nat Med 5:793-802 (1999); Hulett et al. (1999) Nat Med 5:803-9). The heparanase gene is expressed as two mRNA species containing the same open reading frame (Dong et al. (2000) Gene 253:171-178). This relatively slow progress has been due mainly to the instability of the enzyme and the difficulty in designing quantitative assays. The human heparanase gene is unique and the mRNA encodes a putative 65 kDa precursor and a 50 kDa active protein. The heparanase mRNA and protein are preferentially expressed in highly metastatic mouse, rat and human cell lines and in biopsy specimens of human tumors. Over-expression of this heparanase cDNA in low or non-metastatic tumor cells conferred a high metastatic potential in experimental mice, resulting in an increased rate of mortality. Conversely, inhibition of heparanase activity by structural mimics of heparan sulfate was shown to inhibit primary tumor growth, metastasis and vascularity of tumors significantly in animal models (Nakajima et al. (1988) J Cell Biochem 36: 157-67 (1988); Parish et al. (1999) Cancer Res 59: 3433-41; Willenborg et al. (1998) J Immunol 140:3401-5).

Thus, heparanase activity appears to be closely correlated with disease states such as tumor metastasis; inflammatory diseases (e.g., via migration of leukocytes into sites of inflammation) and allograft rejection. Accordingly, there is a need for methods and compositions which allow for specific and targeted modulation of heparanase expression.

### SUMMARY

Described herein are novel heparanase sequences, particularly novel sequences from the regulatory regions upstream and downstream of the coding region. Further, also described are compositions and methods for modulating expression of heparanase. For example, using binding proteins such as zinc finger DNA binding proteins which are targeted to the novel heparanase sequences described herein, novel transcription activator and repressor proteins, which are capable of activating or repressing heparanase gene expression in vivo, are generated. Additionally, chimeric activator and repressor proteins bind to relevant target sites in the heparanase gene and activate or repress heparanase transcription. Polynucleotides encoding these proteins are also provided. Also described are expression constructs which include the polynucleotides described herein or functional fragments thereof. These expression constructs find use, for example, in modulating expression of a target gene. Thus, the compositions and methods described herein provide novel gene therapy approaches for metastatic cancer, inflammatory diseases and the like.

In one aspect, described herein is an isolated polynucleotide comprising a heparanese sequence having X contiguous nucleotides, wherein (i) the X contiguous nucleotides have at least about 80% identity to Y contiguous nucleotides derived from SEQ ID NO:2, (ii) X equals Y, and (iii) X is greater than or equal to 50. In other aspects, described herein is an isolated polynucleotide comprising a heparanese sequence having X contiguous nucleotides, wherein (i) the X contiguous nucleotides have at least about 80% identity to Y contiguous nucleotides derived from SEQ ID NO:3, (ii) X equals Y, and (iii) X is greater than or equal to 50. In additional aspects, described herein is an isolated polynucleotide comprising a heparanese sequence having X contiguous nucleotides, wherein (i) the X contiguous nucleotides have at least about 80% identity to Y contiguous nucleotides derived from SEQ ID NO:18, (ii) X equals Y, and (iii) X is greater than or equal to 50. In certain embodiments of the isolated polynucleotides described herein, X is between about 50 and 650, including all integer values between 50 and 650. In other embodiments of the isolated polynucleotides described herein, X is greater than or equal to 650. In other embodiments, provided herein is an isolated polynucleotide comprising SEQ ID NO:2 or an isolated polynucleotide comprising SEQ ID NO:3 or an isolated polynucleotide comprising SEQ ID NO:18.

In another aspect, an expression vector comprising any of the isolated polynucleotides described herein is provided.

In another aspect, a host cell comprising any of the isolated polynucleotides or expression vectors described herein is provided.

In yet another aspect, a fusion polypeptide comprising (a) a DNA binding domain targeted to a region of any of the isolated polynucleotides described herein; and (b) a transcriptional regulatory domain or functional fragment thereof is provided. In certain embodiments, the DNA binding domain is a zinc finger DNA binding domain. In yet other embodiments, the targeted region is at least 9 nucleotides in length. In still further embodiments, the transcriptional regulatory domain comprises a repression domain, for example, (a) KRAB; (b) MBD2B; (c) v-erbA and (d) functional fragments of (a), (b) or (c). In other embodiments, the transcriptional regulatory domain comprises an activation domain, for example, (a) VP16; (b) p65 and (c) functional fragments of (a) or (b).

In yet another aspect, a polynucleotide encoding any of the fusion polypeptides described herein is provided. Host cells comprising these fusion polypeptides (and polynucleotides encoding these polypeptides) are also provided.

In still another aspect, a method of modulating expression of a heparanase gene is provided. In certain embodiments, the method comprises the step of contacting a region of any of the sequences disclosed herein (e.g., SEQ ID NO:2; SEQ ID NO:3; SEQ ID NO: 18) or fragments thereof with a molecule that binds to a binding site in the region. In other embodiments, the method comprises the step of contacting a region of any of the isolated polynucleotides described herein with a molecule that binds to a binding site in the region. In certain embodiments, the molecule is an endogenous transcriptional regulatory factor. In other embodiments, the molecule comprises a fusion molecule comprising a DNA binding domain and a transcriptional regulatory domain or functional fragments thereof. The region may be any length, and is preferably at least 9 nucleotides in length. In certain embodiments, the modulation comprises repression of heparanase, for example where the transcriptional regulatory domain comprises a repression domain such as (a) KRAB; (b) MBD2B; (c) v-erbA and (d) functional fragments of (a), (b) or (c). In other embodiments, the modulation of the heparanase gene comprises activation of heparanase, for example, where the transcriptional regulatory domain comprises an activation domain such as (a) VP16; (b) p65; and (c) functional fragments of (a) or (b). Any of the methods described herein may be carried out in, for example, a yeast cell, an insect cell, a plant cell or animal cell (e.g, human cell).

In another aspect, described herein is a recombinant expression construct effective in directing the transcription of a selected coding sequence, said expression construct comprising: (a) a coding sequence; and (b) control elements that are operably linked to said coding sequence, wherein said control elements comprise a polynucleotide derived from any of the polynucleotides described herein or a functional fragment thereof, and wherein said coding sequence can be transcribed and translated in a host cell. In other aspects, a host cell transformed with any of the recombinant expression constructs described herein is provided. Also provided is a method of modulating expression of a target coding sequence in a host cell comprising the step of contacting the host cell with any of the expression constructs described herein, wherein the expression construct comprises the target coding sequence.

These and other embodiments will readily occur to those of skill in the art in light of the disclosure herein.

### BRIEF DESCRIPTION OF THE DRAWINGS

FIG. 1 shows the sequence of the first and second exons of the human heparanase gene as determined by Dong et al. (2000) Gene 253:171-178 (SEQ ID NO:1). Exon sequences are in uppercase and are identified to the right of the figure; intron sequences are in lowercase. The translation initiation codon is shown in boldface type. Regions of this sequence used for construction of primers to obtain additional flanking sequence are underlined and the identities of the primers so obtained are given to the right of the figure.

FIG. 2 shows new human heparanase upstream sequence (SEQ ID NO:2) determined as disclosed herein. An additional T residue may be present in the region indicated in boldface. See Example 1.

FIG. 3 shows new human heparanase downstream sequence (SEQ ID NO:3) determined as disclosed herein. See Example 1.

FIG. 4 shows the nucleotide sequence of the human heparanase gene in the vicinity of the upstream region and the first and second exons (SEQ ID NO:4). Numbering is with respect to the translation initiation site, with the A residue of the ATG initiation codon (boxed) designated +1. Exons of the transcript initiated at the upstream transcription initiation site are given in upper case; the unique portion of the exon from the transcript initiated at the downstream transcription initiation site is given in boldface, and regions of the sequence that are DNase hypersensitive in MDA435 cells (see FIG. 5 and Example 2) are underlined.

FIGS. 5A-C show an analysis of DNase hypersensitivity in the upstream and promoter-proximal regions of the human heparanase gene in MDA435 cells. FIG. 5A shows analysis from a Nco I site located downstream of the transcriptional startsites. FIG. 5BB shows analysis from a Hind III site upstream of the transcriptional startsites. For FIGS. 5A and 5B, lanes 1-3 show the products of digestion of chromosomal DNA with increasing concentrations of DNase I, as analyzed by indirect end-labeling with a probe that abutted either the downstream Nco I site (FIG. 5A) or the upstream Hind III site (FIG. 5B). Lanes 4-6 show size markers generated by hybridization of the probe to double digests of chromosomal DNA, as indicated above the lanes. The sizes of the marker fragments are shown to the right of the figures. The results are summarized in FIG. 5C. Shaded boxes indicate the locations of restriction fragments used as probes for indirect end-labeling. Cross-hatched boxes delineate the approximate boundaries of the accessible (DNase-hypersensitive) regions identified in this experiment. Arrows indicate the transcriptional startsites, and locations of restriction sites are indicated. Numbering is with respect to the translation initiation site.

FIG. 6 shows the locations of binding sites for transcription factors within accessible regions of the human heparanase promoter. The nucleotide sequence of the human heparanase gene in the vicinity of the upstream region and the first and second exons (SEQ ID NO:4) is shown. Numbering is with respect to the translation initiation site, with the A residue of the ATG initiation codon (boxed) designated +1. Exons of the transcript initiated at the upstream transcription initiation site are given in upper case; the unique portion of the exon from the transcript initiated at the downstream transcription initiation site is given in boldface, and regions of the sequence that are DNase hypersensitive in MDA435 cells (see FIG. 5 and Example 2) are underlined. Potential transcription initiation sites (as suggested by Dong et al., supra) are indicated by inverted triangles. Binding sites are shaded and the identity of the factor which binds to each particular site is indicated above the site. See Example 4.

FIG. 7 shows levels of heparanase mRNA in various cell lines and tissues. See Example 5.

FIG. 8 shows levels of heparanase mRNA in PC-3 cells that have been transfected with nucleic acids encoding various ZFP-activation domain fusion molecules. The prefix indicates the identity of the activation domain present in the fusion molecule, with “v” indicating VP16 and “s” indicating p65. The identity of the ZFP DNA-binding domain is indicated by the number, which refers to the SBS numbers given in Tables 2 and 3. “NVF” indicates cells that were transfected with a nucleic acid encoding the VP16 activation domain but lacking a ZFP DNA-binding domain. “Non-tf” indicates non-transfected cells. Heparanase mRNA levels were measured and normalized to GAPDH mRNA levels as described in Example 5.

FIG. 9 shows a dose-response analysis of heparanase gene expression in human 293 cells relative to amount of ZFP-encoding nucleic acid transfected. Symbols are the same as in FIG. 8 and the amount of transfected ZFP-encoding nucleic acid is indicated by the shading of the bars.

FIG. 10 presents the nucleotide sequence of a region of the human heparanase gene located upstream of the translation initiation site. The translation initiation site (ATG) is indicated by dark shading. Nuclease hypersensitive regions, as determined in Example 2, are indicated by lighter shading. Restriction enzyme recognition sites are provided above each line of sequence. Within this sequence, target sites for some of the ZFP DNA-binding domains shown in Tables 2 and 3 are shown in bold and underlined, and the SBS number of the ZFP is given below the target site. Note that there are two target site for SBS#519, one of them overlaps with the SBS#1770 target site. Note also that SBS#5349 is a six-finger protein comprising the two 3-finger protein SBS#519 and SBS#1755. Accordingly, the SBS#5349 target site is the composite of the downstream SBS#519 binding site and the SBS#1755 binding site.

FIG. 11 shows the nucleotide sequence of a human heparanase gene regulatory region.

### DETAILED DESCRIPTION

The practice of the disclosed methods and use of the discloses compositions employ, unless otherwise indicated, conventional techniques in molecular biology, biochemistry, genetics, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989; Ausubel et al., CURRENT PROTOCOLS IN MOLECULAR BIOLOGY, John Wiley & Sons, New York, 1987 and periodic updates; and the series METHODS IN ENZYMOLOGY, Academic Press, San Diego.

The disclosures of all patents, patent applications and publications mentioned herein are hereby incorporated by reference in their entireties.

Definitions

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. Thus, the term polynucleotide sequence is the alphabetical representation of a polynucleotide molecule. This alphabetical representation can be input into databases in a computer having a central processing unit and used for bioinformatics applications such as functional genomics and homology searching.

Typical “control elements” include, but are not limited to, transcription promoters, transcription enhancer elements, cis-acting transcription regulating elements (transcription regulators, e.g., a cis-acting element that affects the transcription of a gene, for example, a region of a promoter with which a transcription factor interacts to modulate expression of a gene), transcription termination signals, as well as polyadenylation sequences (located 3′ to the translation stop codon), sequences for optimization of initiation of translation (located 5′ to the coding sequence), translation enhancing sequences, and translation termination sequences. Control elements are preferably derived from the polynucleotides described herein (e.g., heparanase sequences) and include functional fragments thereof, for example, polynucleotides between about 5 and about 50 nucleotides in length (or any integer therebetween); preferably between about 5 and about 25 nucleotides (or any integer therebetween), even more preferably between about 5 and about 10 nucleotides (or any integer therebetween), and most preferably 9-10 nucleotides. Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. When claiming sequences relative to sequences described herein, the range of desired degrees of sequence identity is approximately 80% to 100% and any integer value therebetween. Typically the percent identities between the disclosed sequences and the claimed sequences are at least 70-75%, preferably 80-82%, more preferably 85-90%, even more preferably 92%, still more preferably 95%, and most preferably 98% sequence identity to the reference sequence (i.e., the sequences disclosed herein).

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two DNA, or two polypeptide sequences are “substantially homologous” to each other when the sequences exhibit at least about 70%-75%, preferably 80%-82%, more preferably 85%-90%, even more preferably 92%, still more preferably 95%, and most preferably 98% sequence identity to the reference sequence over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

“Selective hybridization” of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” is a protein or segment within a larger protein that binds DNA in a sequence-specific manner as a result of stabilization of protein structure through coordination of a zinc ion. The term zinc finger DNA binding protein is often abbreviated as zinc finger protein or ZFP.

A “designed” zinc finger protein is a protein not occurring in nature whose design/composition results principally from rational criteria. Rational criteria for design include application of substitution rules and computerized algorithms for processing information in a database storing information of existing ZFP designs and binding data. A “selected” zinc finger protein is a protein not found in nature whose production results primarily from an empirical process such as phage display. See e.g., U.S. Pat. No. 5,789,538; U.S. Pat. No. 6,007,988; U.S. Pat. No. 6,013,453; U.S. Pat. No. 6,140,081; U.S. Pat. No. 6,140,466; WO 95/19431; WO 96/06166 and WO 98/54311.

The term “naturally-occurring” is used to describe an object that can be found in nature, as distinct from being artificially produced by humans.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically joined in cis and can be contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a transcriptional activation domain (or functional fragment thereof), the ZFP DNA-binding domain and the transcriptional activation domain (or functional fragment thereof) are in operative linkage if, in the fusion polypeptide, the ZFP DNA-binding domain portion is able to bind its target site and/or its binding site, while the transcriptional activation domain (or functional fragment thereof) is able to activate transcription.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding function, ability to hybridize to another nucleic acid, binding to a regulatory molecule) are well-known in the art. Similarly, methods for determining protein function are well-known. For example, the DNA-binding function of a polypeptide can be determined, for example, by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

“Specific binding” between, for example, a ZFP and a specific target site means a binding affinity of at least 1×10⁶ M⁻¹.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and a methyl binding domain) and fusion nucleic acids (for example, a nucleic acid encoding a fusion polypeptide). Examples of the second type of fusion molecule include, but are not limited to, a fusion between a triplex-forming nucleic acid and a polypeptide, and a fusion between a minor groove binder and a nucleic acid.

An “exogenous molecule” is a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotien, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (i.e., an exogenous gene), providing it has a sequence that is different from an endogenous molecule. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include endogenous genes and endogenous proteins, for example, transcription factors and components of chromatin remodeling complexes.

A “gene,” for the purposes of the present disclosure, includes a DNA region encoding a gene product (see below), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” and “augmentation of gene expression” refer to any process which results in an increase in production of a gene product. A gene product can be either RNA (including, but not limited to, mRNA, rRNA, tRNA, and structural RNA) or protein. Accordingly, gene activation includes those processes which increase transcription of a gene and/or translation of a mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes which increase translation include those which increase translational initiation, those which increase translational elongation and those which increase mRNA stability. In general, gene activation comprises any detectable increase in the production of a gene product, preferably an increase in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

“Gene repression” and “inhibition of gene expression” refer to any process which results in a decrease in production of a gene product. A gene product can be either RNA (including, but not limited to, mRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more. Most preferably, gene repression results in complete inhibition of gene expression, such that no gene product is detectable.

“Modulation” of gene expression includes both gene activation and gene repression. Modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology 15:961-964); changes in signal transduction; changes in phosphorylation and dephosphorylation; changes in receptor-ligand interactions; changes in concentrations of second messengers such as, for example, cGMP, cAMP, IP₃, and Ca2⁺; changes in cell growth, changes in neovascularization, and/or changes in any functional effect of gene expression. Measurements can be made in vitro, in vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP₃); changes in intracellular calcium levels; cytokine release, and the like.

“Eucaryotic cells” include, but are not limited to, fungal cells (such as yeast), plant cells, animal cells, mammalian cells and human cells.

A “regulatory domain” or “functional domain” refers to a protein or a polypeptide sequence that has transcriptional modulation activity. In one embodiment, a regulatory domain is covalently or non-covalently linked to a ZFP to modulate transcription of a gene of interest. Alternatively, a ZFP can act alone, without a regulatory domain, to modulate transcription. Furthermore, transcription of a gene of interest can be modulated by a ZFP linked to multiple regulatory domains. In addition, a regulatory domain can be linked to any DNA-binding domain having the appropriate specificity to modulate the expression of a gene of interest.

In the context of nucleotide sequences, a “regulatory sequence” or “regulatory region” is a region of sequence which can mediate modulation of gene expression. Modulation of gene expression can occur, for example, if a regulatory region is bound by an appropriate regulatory molecule (or molecules), either endogenous or exogenous.

A “target site” or “target sequence” is a sequence that is bound by a binding protein or binding domain such as, for example, a ZFP. Target sequences can be nucleotide sequences (either DNA or RNA) or amino acid sequences. By way of example, a DNA target sequence for a three-finger ZFP is generally either 9 or 10 nucleotides in length, depending upon the presence and/or nature of cross-strand interactions between the ZFP and the target sequence.

Overview

The compositions and methods disclosed herein include new human heparanase gene sequences, newly-identified regulatory regions of the human heparanase gene, and molecules which regulate gene expression through their interaction with heparanase regulatory sequences. These methods and compositions allow for targeted modulation of expression of the heparanase gene, as well as modulation of expression of a target gene using heparanase regulatory sequences. Compositions include functional domains fused to a DNA-binding domain specific for heparanase regulatory sequences such as, for example, a designed zinc finger DNA binding domain. Modulation of gene expression (e.g., mRNA and protein production) can be determined, for example, in mammalian cells through transient transfection assays and the production of stable cell lines, and/or by measuring the level of heparanase or target gene expression in the absence and presence of the fusion molecules described above.

Modulation of Heparanase Gene Expression

In preferred embodiments, the compositions described herein comprise a binding protein that is targeted to regulatory sequences of a heparanase gene in combination with a transcriptional regulatory domain (or functional fragment thereof). Using these compositions, expression of a target gene, for example, heparanase can be modulated (e.g., repressed or activated) to facilitate targeted control of disease states such as tumor metastasis, inflammatory diseases, allograft rejection and the like.

A. Heparanase Sequences

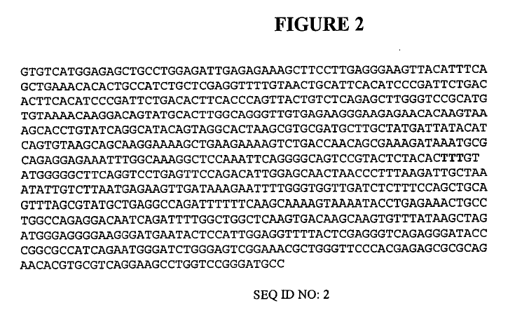

Described herein are novel sequences of human heparanase, particularly sequences from regions upstream and downstream of the two recently-sequenced exons. These novel flanking sequences were obtained as described in Examples 1 and 8, and generally include one or more regulatory elements. The sequences are shown in FIGS. 2, 3 and 11, as well as in SEQ ID Nos:2, 3 and 18.

In preferred embodiments, the novel sequences are least about 80% homologous to at least 50 or more contiguous nucleotides presented in (a) SEQ ID NO:2, (b) SEQ ID NO:3, or (c) SEQ ID NO:18. The novel sequences are preferably between about 50 and 650 base pairs in length (or any integer value therebetween). The novel sequences described herein can be used in construction of expression vectors and can be inserted into host cells, using methods known to those of skill in the art and in view of the teachings herein. Suitable host cells include, but are not limited to, mammalian cells, insect cells, plant cells and yeast cells.

In yet other embodiments, the novel sequences described herein (or functional fragments derived from these sequences) are used to modulate expression of a target coding sequence. Functional fragments are polynucleotides of any length which are able to function as control elements and can be determined by methods known in the art. Typically, control elements will be between about 5 and 50 nucleotides in length, preferably between about 5 and 25 nucleotides in length, more preferably between about 5 and 10 nucleotides in length and most preferably 9-10 nucleotides in length. Thus, expression constructs comprising the novel (e.g., regulatory) sequences or functional fragments thereof operably linked to a coding sequence can be constructed. These constructs can be used, for example, in methods to modulate expression, e.g., by transforming a host cell to obtain regulated expression of the coding sequence.

In additional embodiments, the novel sequences disclosed herein are used for regulation of an endogenous heparanase gene residing in cellular chromatin. In one embodiment, regulation is achieved by using the disclosed heparanase regulatory sequences to guide the design of a regulatory molecule comprising a DNA-binding domain fused to a functional domain. A preferred DNA-binding domain is a zinc finger DNA-binding domain (ZFP). Exemplary functional domains are disclosed infra. In a preferred embodiment, a regulatory molecule is used to inhibit expression of an endogenous heparanase gene, to block tumor metastasis. Such regulatory molecules can also be used to regulate expression of a target gene operatively linked to one or more heparanase regulatory sequences.

B. DNA-Binding Domains

In preferred embodiments, the compositions and methods disclosed herein involve use of DNA binding proteins, particularly zinc finger proteins. A DNA-binding domain can comprise any molecular entity capable of sequence-specific binding to chromosomal DNA. Binding can be mediated by electrostatic interactions, hydrophobic interactions, or any other type of chemical interaction. Examples of moieties which can comprise part of a DNA-binding domain include, but are not limited to, minor groove binders, major groove binders, antibiotics, intercalating agents, peptides, polypeptides, oligonucleotides, and nucleic acids. An example of a DNA-binding nucleic acid is a triplex-forming oligonucleotide.

Minor groove binders include substances which, by virtue of their steric and/or electrostatic properties, interact preferentially with the minor groove of double-stranded nucleic acids. Certain minor groove binders exhibit a preference for particular sequence compositions. For instance, netropsin, distamycin and CC-1065 are examples of minor groove binders which bind specifically to AT-rich sequences, particularly runs of A or T. WO 96/32496.

Many antibiotics are known to exert their effects by binding to DNA. Binding of antibiotics to DNA is often sequence-specific or exhibits sequence preferences. Actinomycin, for instance, is a relatively GC-specific DNA binding agent.

In a preferred embodiment, a DNA-binding domain is a polypeptide. Certain peptide and polypeptide sequences bind to double-stranded DNA in a sequence-specific manner. For example, transcription factors participate in transcription initiation by RNA Polymerase II through sequence-specific interactions with DNA in the promoter and/or enhancer regions of genes. Defined regions within the polypeptide sequence of various transcription factors have been shown to be responsible for sequence-specific binding to DNA. See, for example, Pabo et al. (1992) Ann. Rev. Biochem. 61:1053-1095 and references cited therein. These regions include, but are not limited to, motifs known as leucine zippers, helix-loop-helix (HLH) domains, helix-turn-helix domains, zinc fingers, β-sheet motifs, steroid receptor motifs, bZIP domains homeodomains, AT-hooks and others. The amino acid sequences of these motifs are known and, in some cases, amino acids that are critical for sequence specificity have been identified. Polypeptides involved in other process involving DNA, such as replication, recombination and repair, will also have regions involved in specific interactions with DNA. Peptide sequences involved in specific DNA recognition, such as those found in transcription factors, can be obtained through recombinant DNA cloning and expression techniques or by chemical synthesis, and can be attached to other components of a fusion molecule by methods known in the art.

In a more preferred embodiment, a DNA-binding domain comprises a zinc finger DNA-binding domain. See, for example, Miller et al. (1985) EMBO J. 4:1609-1614; Rhodes et al. (1993) Scientific American Feb.:56-65; and Klug (1999) J. Mol. Biol. 293:215-218. The three-fingered Zif268 murine transcription factor has been particularly well studied. (Pavletich, N. P. & Pabo, C. O. (1991) Science 252:809-17). The X-ray co-crystal structure of Zif268 ZFP and double-stranded DNA indicates that each finger interacts independently with DNA (Nolte et al. (1998) Proc Natl Acad Sci USA 95:2938-43; Pavletich, N. P. & Pabo, C. O. (1993) Science 261:1701-7). The organization of the 3-fingered domain allows recognition of three contiguous base-pair triplets by each finger. Each finger is approximately 30 amino acids long, adopting a ββα fold. The two β-strands form a sheet, positioning the recognition α-helix in the major groove for DNA binding. Specific contacts with the bases are mediated primarily by four amino acids immediately preceding and within the recognition helix. Conventionally, these recognition residues are numbered −1, 2, 3, and 6 based on their positions in the α-helix.

ZFP DNA-binding domains are designed and/or selected to recognize a particular target site as described in co-owned WO 00/42219; WO 00/41566; and WO 98/53057, WO 98/53058, WO 98/53059, and WO 98/53060; as well as U.S. Pat. Nos. 5,789,538; 6,007,408; 6,013,453; 6,140,081; and 6,140,466; and PCT publications WO 95/19431, WO 98/54311, WO 00/23464 and WO 00/27878. In one embodiment, a target site for a zinc finger DNA-binding domain is identified according to site selection rules disclosed in co-owned WO 00/42219. In a preferred embodiment, a ZFP is selected as described in co-owned U.S. Ser. No. 09/716,637.

In certain preferred embodiments, the binding specificity of the DNA-binding domain can be determined by identifying accessible regions in the sequence in question (e.g., in cellular chromatin). Accessible regions can be determined as described in co-owned WO 01/83732, the disclosure of which is hereby incorporated by reference herein. See also Example 2. A DNA-binding domain is then designed and/or selected as described herein to bind to a target site within the accessible region.

C. Fusion Molecules

The identification of novel heparanase sequences and accessible regions (e.g., DNase I hypersensitive sites) in the heparanase gene allows for the design of fusion molecules which facilitate regulation of heparanase gene expression. Thus, in certain embodiments, the compositions and methods disclosed herein involve fusions between a DNA-binding domain specifically targeted to regulatory regions of the heparanase gene and a functional (e.g., repression or activation) domain (or a polynucleotide encoding such a fusion). In this way, the repression or activation domain is brought into proximity with a sequence in the heparanase gene that is bound by the DNA-binding domain. The transcriptional regulatory function of the functional domain is then able to act on heparanase.

In additional embodiments, targeted remodeling of chromatin, as disclosed in co-owned WO 01/83793, can be used to generate one or more sites in cellular chromatin that are accessible to the binding of a heparanese DNA binding molecule.

Fusion molecules are constructed by methods of cloning and biochemical conjugation that are well-known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935.

The fusion molecules disclosed herein comprise a DNA-binding domain which binds to a target site in heparanase. In certain embodiments, the target site is present in an accessible region of cellular chromatin. Accessible regions can be determined as described, for example, in co-owned WO 01/83732. If the target site is not present in an accessible region of cellular chromatin, one or more accessible regions can be generated as described in co-owned WO 01/83793. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3). Cordingley et al. (1987) Cell 48:261-270; Pina et al. (1990) Cell 60:719-731; and Cirillo et al. (1998) EMBO J. 17:244-254.

Methods of gene regulation targeted to a specific sequence with a DNA binding domain can achieve modulation of heparanase gene expression. Modulation of gene expression can be in the form of increased expression or repression. As described herein, repression of heparanase expression can be used to reduce or prevent tumor metastasis and other disease processes. Alternatively, modulation can be in the form of activation, if activation of heparanase is desired. In this case, cellular chromatin is contacted with a fusion molecule comprising, an activation domain and a heparanase DNA-binding domain. Preferably, the DNA-binding domain is specific for a regulatory element of heparanase.

For such applications, the fusion molecule is typically formulated with a pharmaceutically acceptable carrier, as is known to those of skill in the art. See, for example, Remington's Pharmaceutical Sciences, 17^{th} ed., 1985; and co-owned WO 00/42219.

The functional component/domain can be selected from any of a variety of different components capable of influencing transcription of a gene once the exogenous molecule binds to an identified regulatory sequence via the DNA binding domain of the exogenous molecule. Hence, the functional component can include, but is not limited to, various transcription factor domains, such as activators, repressors, co-activators, co-repressors, and silencers.

An exemplary functional domain for fusing with a DNA-binding domain such as, for example, a ZFP, to be used for repressing expression of heparanase is a KRAB repression domain from the human KOX-1 protein (see, e.g., Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Another suitable repression domain is methyl binding domain protein 2B (MBD-2B) (see, also Hendrich et al. (1999) Mamm Genome 10:906-912 for description of MBD proteins). Another useful repression domain is that associated with the v-ErbA protein. See, for example, Damm, et al. (1989) Nature 339:593-597; Evans (1989) Int. J. Cancer Suppl. 4:26-28; Pain et al. (1990) New Biol. 2:284-294; Sap et al. (1989) Nature 340:242-244; Zenke et al. (1988) Cell 52:107-119; and Zenke et al. (1990) Cell 61:1035-1049.

Suitable domains for achieving activation include the HSV VP 16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)).

Additional exemplary activation domains include, but are not limited to, VP16, VP64, p300, CBP, PCAF, SRC1 PvALF, AtHD2A and ERF-2. See, for example, Robyr et al. (2000) Mol. Endocrinol. 14:329-347; Collingwood et al. (1999)J. Mol. Endocrinol. 23:255-275; Leo et al. (2000) Gene 245:1-11; Manteuffel-Cymborowska (1999) Acta Biochem. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Additional exemplary repression domains include, but are not limited to, KRAB, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chern et al. (1996) Plant Cell 8:305-321; and Wu et al. (2000) Plant J. 22:19-27.

Additional functional domains are disclosed, for example, in co-owned WO 00/41566.

Polynucleotide and Polypeptide Delivery

The compositions described herein can be provided to the target cell in vitro or ill vivo. In addition, the compositions can be provided as polypeptides, polynucleotides or combination thereof.

A. Delivery of Polynucleotides

In certain embodiments, the compositions are provided as one or more polynucleotides. Further, as noted above, the compositions described herein may be designed as a fusion between a heparanase DNA-binding domain and a functional domain (e.g., repressive domain) and can be encoded by a fusion nucleic acid. In both fusion and non-fusion cases, the nucleic acid can be cloned into intermediate vectors for transformation into prokaryotic or eukaryotic cells for replication and/or expression. Intermediate vectors for storage or manipulation of the nucleic acid or production of protein can be prokaryotic vectors, (e.g., plasmids), shuttle vectors, insect vectors, or viral vectors for example. A nucleic acid can also cloned into an expression vector, for administration to a bacterial cell, fungal cell, protozoal cell, plant cell, or animal cell, preferably a mammalian cell, more preferably a human cell.

To obtain expression of a cloned nucleic acid, it is typically subcloned into an expression vector that contains a promoter to direct transcription. Suitable bacterial and eukaryotic promoters are well known in the art and described, e.g., in Sambrook et al., supra; Ausubel et al., supra; and Kriegler, Gene Transfer and Expression: A Laboratory Manual (1990). Bacterial expression systems are available in, e.g., E. coli, Bacillus sp., and Salmonella. Palva et al. (1983) Gene 22:229-235. Kits for such expression systems are commercially available. Eukaryotic expression systems for mammalian cells, yeast, and insect cells are well known in the art and are also commercially available, for example, from Invitrogen, Carlsbad, Calif. and Clontech, Palo Alto, Calif.

The promoter used to direct expression of the nucleic acid of choice depends on the particular application. For example, a strong constitutive promoter is typically used for expression and purification. In contrast, when a protein is to be used in vivo, either a constitutive or an inducible promoter is used, depending on the particular use of the protein. In addition, a weak promoter can be used, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system. See, e.g., Gossen et al. (1992) Proc. Natl. Acad. Sci USA 89:5547-5551; Oligino et al. (1998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761.

In addition to a promoter, an expression vector typically contains a transcription unit or expression cassette that contains additional elements required for the expression of the nucleic acid in host cells, either prokaryotic or eukaryotic. A typical expression cassette thus contains a promoter operably linked, e.g., to the nucleic acid sequence, and signals required, e.g., for efficient polyadenylation of the transcript, transcriptional termination, ribosome binding, and/or translation termination. Additional elements of the cassette may include, e.g., enhancers, and heterologous spliced intronic signals.

The particular expression vector used to transport the genetic information into the cell is selected with regard to the intended use of the resulting polypeptide, e.g., expression in plants, animals, bacteria, fungi, protozoa etc. Standard bacterial expression vectors include plasmids such as pBR322, pBR322-based plasmids, pSKF, pET23D, and commercially available fusion expression systems such as GST and LacZ. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High-yield expression systems are also suitable, such as baculovirus vectors in insect cells, for example under the transcriptional control of the polyhedrin promoter or any other strong baculovirus promoter.

Elements that are typically included in expression vectors also include a replicon that functions in E. coli (or in the prokaryotic host, if other than E. coli), a selective marker, e.g., a gene encoding antibiotic resistance, to permit selection of bacteria that harbor recombinant plasmids, and unique restriction sites in nonessential regions of the vector to allow insertion of recombinant sequences.

Standard transfection methods can be used to produce bacterial, mammalian, yeast, insect, or other cell lines that express large quantities of proteins, which can be purified, if desired, using standard techniques. See, e.g., Colley et al. (1989) J. Biol. Chem. 264:17619-17622; and Guide to Protein Purification, in Methods in Enzymology, vol. 182 (Deutscher, ed.) 1990. Transformation of eukaryotic and prokaryotic cells are performed according to standard techniques. See, e.g., Morrison (1977) J. Bacteriol. 132:349-351; Clark-Curtiss et al. (1983) in Methods in Enzymology 101:347-362 (Wu et al., eds).

Any procedure for introducing foreign nucleotide sequences into host cells can be used. These include, but are not limited to, the use of calcium phosphate transfection, DEAE-dextran-mediated transfection, polybrene, protoplast fusion, electroporation, lipid-mediated delivery (e.g., liposomes), microinjection, particle bombardment, introduction of naked DNA, plasmid vectors, viral vectors (both episomal and integrative) and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

Conventional viral and non-viral based gene transfer methods can be used to introduce nucleic acids into mammalian cells or target tissues. Such methods can be used to administer nucleic acids encoding reprogramming polypeptides to cells in vitro. Preferably, nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For reviews of gene therapy procedures, see, for example, Anderson (1992) Science 256:808-813; Nabel et al. (1993) Trends Biotechnol. 11:211-217; Mitani et al. (1993) Trends Biotechnol. 11: 162-166; Dillon (1993) Trends Biotechnol. 11: 167-175; Miller (1992) Nature 357:455460; Van Brunt (1988) Biotechnology 6(10): 1149-1154; Vigne (1995) Restorative Neurology and Neuroscience 8:35-36; Kremer et al (1995) British Medical Bulletin 51(1):31-44; Haddada et al., in Current Topics in Microbiology and immunology, Doerfler and Böhm (eds), 1995; and Yu et al. (1994) Gene Therapy 1:13-26.

Methods of non-viral delivery of nucleic acids include lipofection, microinjection, ballistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386; 4,946,787; and 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Nucleic acid can be delivered to cells (ex vivo administration) or to target tissues (in vivo administration).

The preparation of lipid:nucleic acid complexes, including targeted liposomes such as immunolipid complexes, is well known to those of skill in the art. See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:48174820; and U.S. Pat. Nos. 4,186,183; 4,217,344; 4,235,871; 4,261,975; 4,485,054; 4,501,728; 4,774,085; 4,837,028 and 4,946,787.

The use of RNA or DNA virus-based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, wherein the modified cells are administered to patients (ex vivo). Conventional viral based systems for the delivery of ZFPs include retroviral, lentiviral, poxyiral, adenoviral, adeno-associated viral, vesicular stomatitis viral and herpesviral vectors. Integration in the host genome is possible with certain viral vectors, including the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, allowing alteration and/or expansion of the potential target cell population. Lentiviral vectors are retroviral vector that are able to transduce or infect non-dividing cells and typically produce high viral titers. Selection of a retroviral gene transfer system would therefore depend on the target tissue. Retroviral vectors have a packaging capacity of up to 6-10 kb of foreign sequence and are comprised of cis-acting long terminal repeats (LTRs). The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof. Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; and PCT/US94/05700).

Adeno-associated virus (AAV) vectors are also used to transduce cells with target nucleic acids, e.g., in the in vitro production of nucleic acids and peptides, and for iii vivo and ex vivo gene therapy procedures. See, e.g., West et al. (1987) Virology 160:38-47; U.S. Pat. No. 4,797,368; WO 93/24641; Kotin (1994) Hum. Gene Ther. 5:793-801; and Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin, et al. (1984) Mol. Cell. Biol. 4:2072-2081; Hermonat et al. (1984) Proc. Natl. Acad. Sci. USA 81:6466-6470; and Samulski et al. (1989) J. Virol. 63:3822-3828.

Recombinant adeno-associated virus vectors based on the defective and nonpathogenic parvovirus adeno-associated virus type 2 (AAV-2) are a promising gene delivery system. Exemplary AAV vectors are derived from a plasmid containing the AAV 145 bp inverted terminal repeats flanking a transgene expression cassette. Efficient gene transfer and stable transgene delivery due to integration into the genomes of the transduced cell are key features for this vector system. Wagner et al. (1998) Lancet 351 (9117):1702-3; and Kearns et al. (1996) Gene Ther. 9:748-55.

pLASN and MFG-S are examples are retroviral vectors that have been used in clinical trials. Dunbar et al. (1995) Blood 85:3048-305; Kohn et al. (1995) Nature Med. 1:1017-102; Malech et al. (1997) Proc. Natl. Acad. Sci. USA 94:12133-12138. PA317/pLASN was the first therapeutic vector used in a gene therapy trial. (Blaese et al. (1995) Science 270:475-480. Transduction efficiencies of 50% or greater have been observed for MFG-S packaged vectors. Ellem et al. (1997) Immunol Immunother. 44(1):10-20; Dranoff et al. (1997) Hum. Gene Ther. 1: 111-2.

In applications for which transient expression is preferred, adenoviral-based systems are useful. Adenoviral based vectors are capable of very high transduction efficiency in many cell types and are capable of infecting, and hence delivering nucleic acid to, both dividing and non-dividing cells. With such vectors, high titers and levels of expression have been obtained. Adenovirus vectors can be produced in large quantities in a relatively simple system.

Replication-deficient recombinant adenovirus (Ad) vectorscan be produced at high titer and they readily infect a number of different cell types. Most adenovirus vectors are engineered such that a transgene replaces the Ad E1a, E1b, and/or E3 genes; the replication defector vector is propagated in human 293 cells that supply the required E1 functions in trans. Ad vectors can transduce multiple types of tissues in vivo, including non-dividing, differentiated cells such as those found in the liver, kidney and muscle. Conventional Ad vectors have a large carrying capacity for inserted DNA. An example of the use of an Ad vector in a clinical trial involved polynucleotide therapy for antitumor immunization with intramuscular injection. Sterman et al. (1998) Hum. Gene Ther. 7:1083-1089. Additional examples of the use of adenovirus vectors for gene transfer in clinical trials include Rosenecker et al. (1996) Infection 24:5-10; Sterman et al., supra; Welsh et al. (1995) Hum. Gene Ther. 2:205-218; Alvarez et al. (1997) Hum. Gene Ther. 5:597-613; and Topf et al. (1998) Gene Ther. 5:507-513.

Packaging cells are used to form virus particles that are capable of infecting a host cell. Such cells include 293 cells, which package adenovirus, and Ψ2 cells or PA317 cells, which package retroviruses. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. Missing viral functions are supplied in trans, if necessary, by the packaging cell line. For example, AAV vectors used in gene therapy typically only possess ITR sequences from the AAV genome, which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment, which preferentially inactivates adenoviruses.

In many gene therapy applications, it is desirable that the gene therapy vector be delivered with a high degree of specificity to a particular tissue type. A viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751 reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., F_{ab} or Fᵥ) having specific binding affinity for virtually any chosen cellular receptor. Although the above description applies primarily to viral vectors, the same principles can be applied to non-viral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described infra. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art. See, e.g., Freshney et al., Culture of Animal Cells, A Manual of Basic Technique, 3rd ed., 1994, and references cited therein, for a discussion of isolation and culture of cells from patients.

In one embodiment, hematopoietic stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ stem cells in vitro into clinically important immune cell types using cytokines such a GM-CSF, IFN-γ and TNF-α are known. Inaba et al. (1992) J. Exp. Med. 176:1693-1702.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells). See Inaba et al., supra.

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions, as described below. See, e.g., Remington's Pharmaceutical Sciences, 17th ed., 1989.

B. Delivery of Polypeptides

In other embodiments, for example in certain in vitro situations, the target cells are cultured in a medium containing a functional domain (or functional fragments thereof) fused to a heparanase DNA binding domain.

An important factor in the administration of polypeptide compounds is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins, lipids and other compounds, which have the ability to translocate polypeptides across a cell membrane, have been described.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Autennapedia., was found to be the third helix of the protein, from amino acid position 43 to 58. Prochiantz (1996) Curr. Opin. Neurobiol. 6:629-634. Another subsequence, the h (hydrophobic) domain of signal peptides, was found to have similar cell membrane translocation characteristics. Lin et al. (1995) J. Biol. Chem. 270:14255-14258.

Examples of peptide sequences which can be linked to heparanase targeted functional polypeptide for facilitating its uptake into cells include, but are not limited to: an 11 amino acid peptide of the tat protein of HIV: a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). Other suitable chemical moieties that provide enhanced cellular uptake can also be linked, either covalently or non-covalently, to the polypeptides described herein.

Toxin molecules also have the ability to transport polypeptides across cell membranes. Often, such molecules (called “binary toxins”) are composed of at least two parts: a translocation or binding domain and a separate toxin domain. Typically, the translocation domain, which can optionally be a polypeptide, binds to a cellular receptor, facilitating transport of the toxin into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193.

Such subsequences can be used to translocate polypeptides, including the polypeptides as disclosed herein, across a cell membrane. This is accomplished, for example, by derivatizing the fusion polypeptide with one of these translocation sequences, or by forming an additional fusion of the translocation sequence with the fusion polypeptide. Optionally, a linker can be used to link the fusion polypeptide and the translocation sequence. Any suitable linker can be used, e.g., a peptide linker.

A suitable polypeptide can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer is degraded over time through the action of various agents in the body. Alternatively, active drug release involves using an agent to induce a permeability change in the liposome vesicle. Liposome membranes can be constructed so that they become destabilized when the environment becomes acidic near the liposome membrane. See, e.g., Proc. Natl. Acad. Sci. USA 84:7851 (1987); Biochemistry 28:908 (1989). When liposomes are endocytosed by a target cell, for example, they become destabilized and release their contents. This destabilization is termed fusogenesis. Dioleoylphosphatidylethanolamine (DOPE) is the basis of many “fusogenic” systems.

For use with the methods and compositions disclosed herein, liposomes typically comprise a fusion polypeptide as disclosed herein, a lipid component, e.g., a neutral and/or cationic lipid, and optionally include a receptor-recognition molecule such as an antibody that binds to a predetermined cell surface receptor or ligand (e.g., an antigen). A variety of methods are available for preparing liposomes as described in, e.g.; U.S. Pat. Nos. 4,186,183; 4,217,344; 4,235,871; 4,261,975; 4,485,054; 4,501,728; 4,774,085; 4,837,028; 4,235,871; 4,261,975; 4,485,054; 4,501,728; 4,774,085; 4,837,028; 4,946,787; PCT Publication No. WO 91/17424; Szoka et al. (1980) Ann. Rev. Biophys. Bioeng. 9:467; Deamer et al. (1976) Biochim. Biophys. Acta 443:629-634; Fraley, et al. (1979) Proc. Natl. Acad. Sci. USA 76:3348-3352; Hope et al. (1985) Biochim. Biophys. Acta 812:55-65; Mayer et al. (1986) Biochim. Biophys. Acta 858:161-168; Williams et al. (1988) Proc. Natl. Acad. Sci. USA 85:242-246; Liposomes, Ostro (ed.), 1983, Chapter 1); Hope et al. (1986) Chem. Phys. Lip. 40:89; Gregoriadis, Liposome Technology (1984) and Lasic, Liposomes: from Physics to Applications (1993). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it may be desirable to target a liposome using targeting moieties that are specific to a particular cell type, tissue, and the like. Targeting of liposomes using a variety of targeting moieties (e.g., ligands, receptors, and monoclonal antibodies) has been previously described. See, e.g., U.S. Pat. Nos. 4,957,773 and 4,603,044.

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV-1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes are used. These methods generally involve the incorporation into liposomes of lipid components, e.g., phosphatidylethanolamine, which can be activated for attachment of targeting agents, or incorporation of derivatized lipophilic compounds, such as lipid derivatized bleomycin. Antibody targeted liposomes can be constructed using, for instance, liposomes which incorporate protein A. See Renneisen et al. (1990) J. Biol. Chem. 265:16337-16342 and Leonetti et al. (1990) Proc. Natl. Acad. Sci. USA 87:2448-2451.

Pharmaceutical Compositions and Administration

Heparanese-targeted DNA binding domains (e.g., a zinc finger protein (ZFP)) and functional domains as disclosed herein, and expression vectors encoding these polypeptides, can be used in conjunction with various methods of gene therapy to facilitate the action of a therapeutic gene product. In such applications, the ZFP can be administered directly to a patient to facilitate the modulation of gene expression and for therapeutic or prophylactic applications, for example, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cardiovascular disease, cystic fibrosis, stroke, and the like. Examples of microorganisms whose replication and/or pathogenicity can be inhibited through use of the methods and compositions disclosed herein include pathogenic bacteria, e.g., Chlamydia, Rickettsial bacteria, Mycobacteria, Staphylococci, Streptococci, Pneumococci, Meningococci and Conococci, Klebsiella, Proteus, Serratia, Pseudomonas, Legionella, Diphtheria, Salmonella, Bacilli (e.g., anthrax), Vibrio (e.g., cholera), Clostridium (e.g., tetanus, botulism), Yersinia (e.g., plague), Leptospirosis, and Borrellia (e.g., Lyme disease bacteria); infectious fungus, e.g., Aspergillus, Candida species; protozoa such as sporozoa (e.g., Plasmodia), rhizopods (e.g., Entamoeba) and flagellates (Trypanosoma, Leishmania, Trichomonas, Giardia, etc.); viruses, e.g., hepatitis (A, B, or C), herpes viruses (e.g., VZV, HSV-1, HHV-6, HSV-II, CMV, and EBV), HIV, Ebola, Marburg and related hemorrhagic fever-causing viruses, adenoviruses, influenza viruses, flaviviruses, echoviruses, rhinoviruses, coxsackie viruses, cornaviruses, respiratory syncytial viruses, mumps viruses, rotaviruses, measles viruses, rubella viruses, parvoviruses, vaccinia viruses, HTLV viruses, retroviruses, lentiviruses, dengue viruses, papillomaviruses, polioviruses, rabies viruses, and arboviral encephalitis viruses, etc.

Administration of therapeutically effective amounts of heparanase-regulatory polypeptides or nucleic acids encoding these fusion polypeptides is by any of the routes normally used for introducing polypeptides or nucleic acids into ultimate contact with the tissue to be treated. The polypeptides or nucleic acids are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions. See, e.g., Remington's Pharmaceutical Sciences, 17^{th} ed. 1985.

Polypeptides or nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally. The formulations of compounds can be presented in unit-dose or multi-dose sealed containers, such as ampoules and vials. Injection solutions and suspensions can be prepared from sterile powders, granules, and tablets of the kind known to those of skill in the art.

Applications

The compositions and methods disclosed herein can be used to facilitate or inhibit a number of processes involving heparanase activity. These processes include, but are not limited to, tumor metastasis, angiogenesis, cell migration, degradation of the basement membrane and/or extracellular matrix, and allograft rejection. Accordingly, the methods and compositions disclosed herein can be used to affect any of these processes, as well as any other process which can be influenced by heparanase activity.

In preferred embodiments, a functional domain/heparanase DNA-binding domain fusion is used to achieve targeted repression of heparanase. Targeting is based upon the specificity of the DNA-binding domain.

Thus, the methods and compositions disclosed herein can be used in processes such as, for example, therapeutic regulation of heparanase-related disease states, and pharmaceutical discovery (including target discovery, target validation and engineering of cells for high throughput screening methods).

Regulatory Regions

The methods and compositions disclosed herein are useful in the identification of sequences which regulate the expression of the heparanase gene. Without wishing to be bound by any particular theory, it is believed that at least one mechanism by which such regulatory sequences mediate modulation of gene expression is by serving as binding site(s) for transcriptional regulatory molecules. Examples of transcriptional regulatory molecules include, but are not limited to, activators, coactivators, repressors, corepressors, components of chromatin remodeling complexes, methyl binding proteins, and any other molecules which can affect the structure and/or function of a nucleotide sequence to which they bind.

An exemplary method for the identification of a regulatory region for a particular gene is to determine the location(s) of accessible region(s) in or in the vicinity of the gene of interest. See Example 2. A preferred method for identifying accessible regions is by heightened sensitivity to nucleases (e.g., DNaseI) iii vivo. Methods for identification of these so-called nuclease hypersensitive regions, as well as additional methods for the identification of accessible regions, are disclosed in co-owned WO 01/83732, the disclosure of which is hereby incorporated by reference in its entirety.

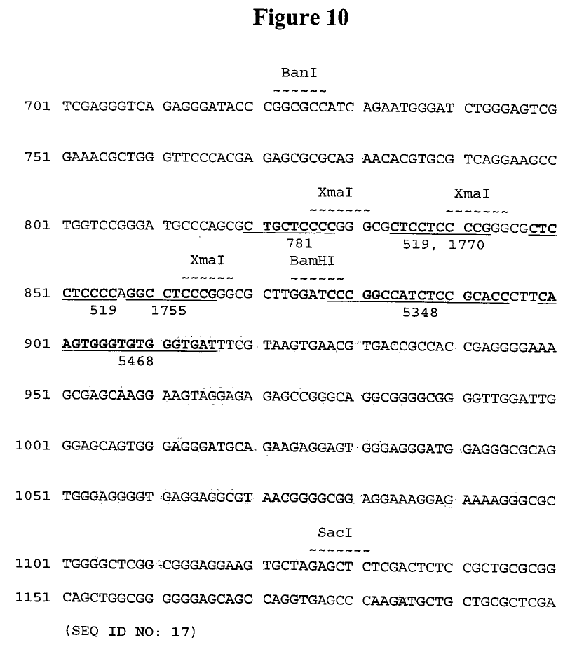

An additional exemplary method for identification of a regulatory region is to design a series of fusion molecules, each of which comprises an activation domain and a DNA-binding domain targeted to a different region in or in the vicinity of the gene. A preferred DNA-binding domain is a zinc finger domain. Additional components of such fusion molecules can include epitope tags and/or nuclear localization signals. The molecules are introduced into cells and expression of the gene of interest is measured. Sequences which serve as targets for the DNA-binding domain in cells in which gene expression is activated are identified as regulatory regions. See Example 7 and FIG. 10. Conversely, sequences which serve as targets for the DNA binding domain of a fusion molecule in cells in which gene expression is not activated are unlikely to be regulatory regions. A similar analysis can be conducted with DNA-binding domain/repression domain fusions; in this embodiment, sequences which serve as targets for the DNA-binding domain in cells in which gene expression is repressed are likely to represent regulatory regions.

Accessible regions of cellular chromatin are believed to be free of nucleosomes, and may in some cases be bound by non-nucleosomal regulatory proteins. Nonetheless, regions of cellular chromatin accessible for binding by a regulatory molecule may extend beyond those that are preferentially sensitive to nucleases. One (but not the only) reason for this is that the size of the nuclease molecule itself precludes its ability to digest the outermost boundaries of an accessible, non-nucleosomal region. In addition, it is possible that chromosomal proteins bound to non-nucleosomal DNA are able to block nuclease access but nonetheless can be displaced by other DNA-binding proteins. Accordingly, in certain embodiments, it is useful to combine the results of both of the above methods to identify regulatory regions. See Example 8.

The following examples are presented as illustrative of, but not limiting, the claimed subject matter.

### EXAMPLES

### Example 1

### Determination of Nucleotide Sequences in the Human Heparanase Gene and Flanking Regions

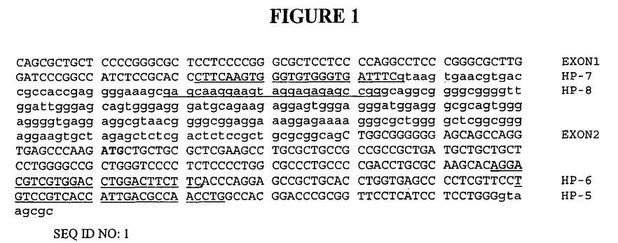

A single heparanase gene is located on human chromosome 4. This gene is expressed as two mRNA species containing the same open reading frame. A 579-nucleotide partial sequence of the human heparanase gene, containing parts of the first and second exons, has been reported by Dong et al. (2000) Gene 253:171-178. This sequence is shown in FIG. 1 (SEQ ID NO 1).

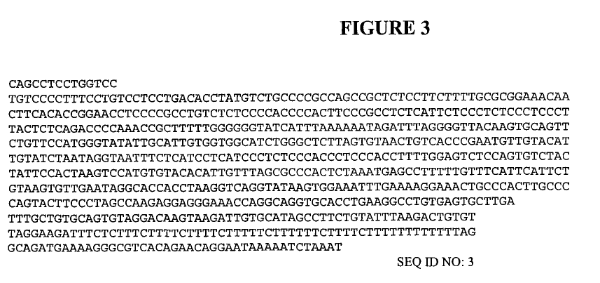

Additional human heparanase gene sequences were obtained by cloning sequences adjacent, on both sides, to the known sequence using a GenomeWalker Kit (Clontech, Palo Alto, Calif.) with MasterAmp Taq DNA polymerase and selected MasterAmp PCR buffers (Epicentre). The heparanase gene-specific primers used in this method are indicated in FIG. 1 and Table 1. The cloned products were sequenced, to obtain approximately 800 bp of new upstream genomic sequence (FIG. 2, SEQ ID NO: 2), and approximately 900 bp of new downstream genomic sequence (FIG. 3, SEQ ID NO: 3).

Primary PCR reactions were performed in a 20 μl reaction volume containing IX MasterAmp (Epicentre) buffer D, E or F, 0.6 units MasterAmp Taq DNA polymerase, 0.4 μl of library template, 0.2 μm AP 1 primer (from the GenomeWalker kit) and 0.2 μM of either HP-6 or HP-8 primer (FIG. 1, Table 1). For each of the two gene-specific primers (HP-6 and HP-8) four separate reactions were conducted, each one containing a different of the four library templates provided in the GenomeWalker kit. Touchdown PCR reactions were conducted in a PE9700 thermal cycler (PE BioSystems, Foster City, Calif.) using the following cycling conditions: 7 cycles of 94° C. for 20 sec followed by 72° C. for 5 min; 32 cycles of 94° C. for 20 sec followed by 67° C. for 5 min, then a 67° C. hold for 10 min. Eight microliters of each sample were analyzed on a 1% agarose gel.

For the downstream genomic DNA (obtained in reactions in which HP-6 was used as the gene-specific primer), the primary PCR reaction using the SspI-digested library template in MasterAmp buffer D yielded a single product with a length of ˜900 bp, which was subcloned directly into pCR2.1 (Invitrogen).

Because the primary amplification of upstream genomic DNA did not yield a unique amplification product, upstream genomic DNA was re-amplified using the same conditions as in the primary reaction, except that nested primers were used, and only a single MasterAmp buffer (D or F) was used. The nested primers were AP2 (GenomeWalker) and HP-7 (FIG. 1, Table 1). Secondary amplifications using all four of the GenomeWalker libraries each yielded a major PCR product, which ranged in size from 400 to 950 bp. The 950 bp EcoRV library/buffer F product was gel-purified and subcloned into pCR2.1.

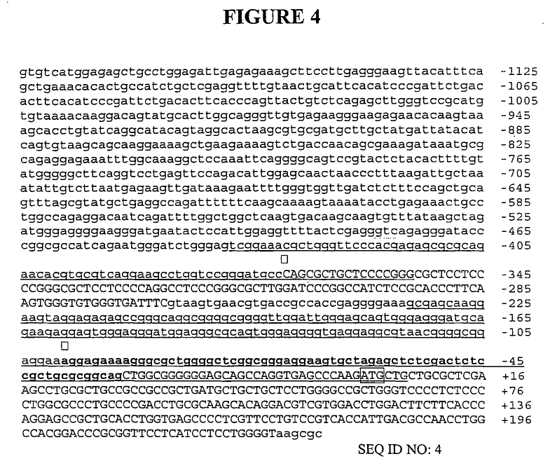

Three colonies from each transformation were checked for insert size; 3/3 were correct for the upstream clones (AP2-HP7) and 1/3 had the insert for the downstream clone (AP1-HP6). One clone from each was sequenced from both ends using the T7 primer, and the M13 reverse primer (Invitrogen). The sequence of the upstream clone is shown in FIG. 2 (SEQ ID NO: 2). The sequence of the downstream clone is shown in FIG. 3 (SEQ ID NO: 2). In FIG. 4, the newly-determined heparanase sequences disclosed herein are combined with existing cDNA sequences to provide an extended human heparanase genomic sequence (SEQ ID NO: 4).

TABLE 1

Human Heparanase Gene-Specific Primers*

| | | SEQ | |
|---|---|---|---|
| | | ID | Rest. |
| Name | Sequence | NO. | site |
| HP-5 | 5′-cataTGTCCGTCACCATTGACGCCAACCTG | 5 | Nde I |
| HP-6 | 5′-ctgcAGGACGTCGTGGACCTGGACTTCTTC | 6 | Pst I |
| HP-7 | 5′-tcgCGAAATCACCCACACCCACTTGAAG | 7 | Nru I |
| HP-8 | 5′-gCCGGCTCTCTCCTACTTCCTTGCTC | 8 | Nae I |

*uppercase letters denote heparanase sequence, lowercase letters are flanking sequences used to generate a restriction site, as indicated in the Table

### Example 2

### Determination of Accessible Regions in the Heparanase Gene

Cell Growth

MDA435 cells, a metastatic breast tumor line, were grown to confluence in DMEM+10% fetal calf serum. Three T225 flasks of confluent cells were washed twice with cold PBS and the cells were scraped into 10 ml cold PBS. The flask was then rinsed with 10 ml cold PBS, which was added to the washes, and the pooled material was centrifuged at 1400 rpm for 5 min. Cells were permeabilized by resuspending the cell pellet in 1.5 ml DNAse I buffer (10 mM Tris-HCl pH 7.5, 10 mM NaCl, 60 mM KCl, 5 mM MgCl₂, 1 mM CaCl, 0.5% Igepal), on ice.

DNase Digestion

A 60 μg/ml solution of DNase I (Worthington, Freehold, N.J.) was made in 1.2 ml of DNase I buffer (supra), then serial 2-fold dilutions were made in DNase I buffer to 30 and 15 μg/ml. Aliquots (0.5 ml) of the resuspended cell pellet from above (permeabilized cell suspension) were equilibrated to room temperature, and to each was added 500 μl of a DNase I solution at 0, 15, 30 or 60 μg/ml, to give final DNase I concentrations of 0, 7.5, 15 and 30 μg/ml. The digestion reactions were then incubated at room temperature for 6 min. Reactions were stopped by the addition of 20 μl of EDTA/RNase A solution (150 μl 0.5 M EDTA, 50 μl 10 mg/ml RNase A) and incubation at room temperature for 5 min. Digested DNA was purified from two 0.2 ml samples, using DNeasy spin filters (Qiagen, Valencia, Calif.) according to the manufacturer's instructions. The remainder of each reaction (0.6 ml) was frozen.

Hypersensitive Site Mapping

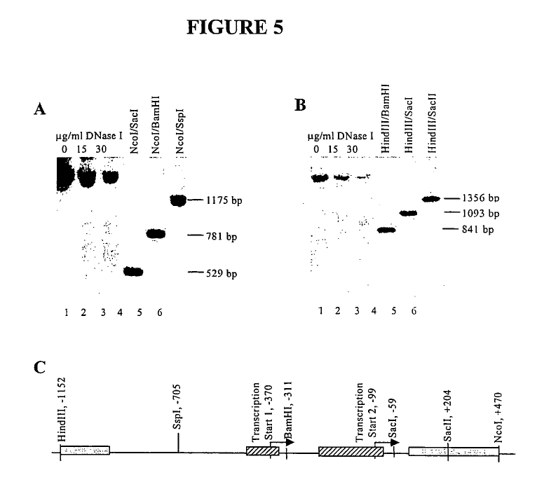

Duplicate samples of DNase i-digested DNA, treated and purified as described above, were digested with either HindIII or NcoI, separated on 1.5% agarose gels (SeaKem GTG, FMC Bioproducts, Rockland, Me.) in TBE buffer, then alkaline-transferred to nylon membranes (Nytran, Schleicher & Schuell, Keene, N.H.). One of the blots was probed with a 212 bp HindIII-EcoN1 fragment which abuts a HindIII site upstream of the heparanase gene. The other blot was probed with a NcoI-BamHI fragment of approximately 400 bp that abuts a NcoI site in the coding region of the heparanase gene. See FIG. 5C. Twenty-five ng of each of these fragments was labeled using a Redi-Prime II kit (Amersham Pharmacia Biotech, Piscataway, N.J.). Blots were exposed to the labeled probes in 15 ml RapidHyb (Amersham Pharmacia Biotech, Piscataway, N.J.) for 2 hours, then washed twice in 0.1×SSC, 0.1% SDS at 65° C. (20 minutes each wash) and exposed overnight to a PhosphorImager screen (Molecular Dynamics, Sunnyvale, Calif.). Results are presented in FIGS. 5A and 5B, and summarized in FIG. 5C.

These results indicated that, in MDA435 cells, two regions of the heparanase gene exhibit enhanced sensitivity to DNase I. These hypersensitive regions are located between −437 and −340 and between −234 and +6, with respect to the translational startsite. Putative transcriptional startsites for the heparanase gene are located at −370 and −99; both of which lie within a hypersensitive region. Similar experiments conducted with Jeg-3 cells, which express 30-fold less heparanase mRNA that do MDA435 cells (see Example 5 below), did not reveal the presence of DNase hypersensitive regions in the chromatin of Jeg-3 calls. The enhanced nuclease sensitivity of these chromosomal regions in cells expressing high levels of heparanase mRNA, taken together with their relationship to the probable transcription startsites, suggest that sequences in these regions are likely to be important for regulation of transcription of the heparanase gene.

### Example 3

### Design of Zinc Finger Proteins Which Bind to the Heparanase Gene

Zinc finger DNA-binding domains, specific for human heparanase sequences, were designed, and their binding constants measured, according to methods disclosed in co-owned WO 00/41566, WO 00/42219, and references disclosed in those publications. The target sequences were chosen to lie within or adjacent to the regions of the heparanase gene that exhibit enhanced sensitivity to DNase I, as determined in Example 2, above. Amino acid sequences of the recognition helices of these proteins, DNA sequences and locations of the target sites, and binding affinity (K_{d}) measurements are presented in Table 2.

Briefly, a PCR-based assembly procedure was used to construct the coding region of the designed zinc finger proteins. For each 3-finger protein, six overlapping oligonucleotides were synthesized. Three of these oligonucleotides (oligos 1, 3, and 5) correspond to the sequences that encode portions of the scaffold for the DNA-binding domain (i.e., portions of the DNA binding domain located between recognition helices) and are constant in different constructs. The other three oligonucleotides (oligos 2, 4, and 6) are designed to encode the recognition helices and thus will vary according to the amino acid sequences required for recognition of the target sequence. These six overlapping oligos were used to construct the “core” of the gene that expresses the ZFP. Then, a pair a external primers (F primer and R primer) with flanking restriction sites compatible for cloning in mammalian and bacterial expression vectors were used to amplify the full lenth synthetic gene. The assembled gene was cloned into pMAL-c2 (New England Biolabs, Beverly, Mass.), generating an in-frame fusion between the HPA-ZFP and malE gene. This created an N-teminal maltose-binding protein (MBP) fusion with the HPA-ZFP. The region encoding the ZFP was sequenced to confirm its acuracy.

Fusion of a ZFP DNA-binding domain with the MBP allowed simple purification and detection of the recombinant protein. ZFP-MBP fusions can be expressed from the pMAL vector in soluble form to high levels in E. coli, and can bind efficiently to their DNA target site without refolding. Liu et al. (1997) Proc. Natl. Acad. Sci. USA 94:5525-5530. Production and purification of MBP-fusion proteins were performed using existing protocols. See, for example, New England BioLabs technical manuals. Purified proteins were examined by SDS-PAGE on a 4-12% gradient gel.

Purified ZFP-MBP fusions were tested for their affinities for their DNA target sites using a quantitative electrophoretic mobility shift assay (EMSA). The target DNA sequences were incorporated into oligonucleotides and assayed using procedures described by Jamieson et al. (1994) Biochemistry 33:5689-5695 and Jamieson et al. (1996) Proc. Natl. Acad. Sci. USA 93:12834-12839. Heparanase DNA target sequences for the EMSA experiments were generated by embedding the 9 bp binding sites within a 30 bp duplex oligonucleotides. Complementary oligonucleotides were synthesized, annealed, and end-labeled with polynucleotide kinase and γ-³²P ATP. Binding affinity of the ZFPs to target oligonucleotides was tested by titrating protein (usually in two-fold serial dilutions) against a fixed amount of substrate oligonucleotide. Twenty-microliter binding reactions contained 50 pM 5′γ-³²P labeled double-stranded target DNA, 10 mM Tris HCl (pH 7.5), 100 mM KCl, 1 mM MgCl₂, 1 mM dithiothreitol, 10% glycerol, 200 ug/ml bovine serum albumin, 0.02% NP-40, and 100 uM ZnCl₂. Binding was allowed to proceed for 45 minutes at room temperature. Polyacrylamide gel electrophoresis was carried out at room temperature using precast 10-20% Tris-HCl gels (BioRad, Hercules, Calif.) and Tris-Glycine running buffer (25 mM Tris HCl, 192 mM glycine, pH 8.3). Radioactive signals were quantitated with a Phosphorimager and by autoradiography. Dissociation constants (K_{d}) were determined to be the protein concentration providing half-maximal binding to the target oligonucleotide, as assayed by altered mobility of bound oligonucleotide. Results of this analysis are presented in Table 2 for ZFPs designed to recognize DNA sequences in the 5′ untranslated region of the heparanase gene, showing that ZFPs with subnanomolar affinities have been obtained.

TABLE 2

Three-finger ZFPs with target sites in the human heparanase gene

| | | | |
|---|---|---|---|
| Target | Target | ZFP Helix³ | K_{d} |

| | | | | | | | |
|---|---|---|---|---|---|---|---|
| SBS#¹ | RS⁵ | Location² | sequence | Finger1 | Finger2 | Finger3 | (nM)⁴ |

| | | | | | | | |
|---|---|---|---|---|---|---|---|
| 781 | Minus | −357 | GGGGAGCAG | RSSNLRE | RSDNLAR | RSDHLTR | 4.25 |
| | | | (SEQ ID NO: 19) | (35) | (36) | (37) | |
| 779 | Minus | −356 | CGGGGAGCA | QSGSLTR | QSGHLTR | RSDHLAE | 0.5 |
| | | | (SEQ ID NO: 20) | (38) | (39) | (40) | |
| 519 | Minus | −343 | GGGGAGGAG | RSDNLTR | RSDNLAR | RSDHLSR | 1.125 |
| | | | (SEQ ID NO: 21) | (41) | (42) | (43) | |
| 1756 | Minus | −342 | CGGGGAGGA | QQAHLAR | QSGHLQR | RSDHLRE | 0.125 |
| | | | (SEQ ID NO: 22) | (44) | (45) | (46) | |
| 519 | Minus | −329 | GGGGAGGAG | RSDNLTR | RSDNLAR | RSDHLSR | 1.125 |
| | | | (SEQ ID NO: 23) | (47) | (48) | (49) | |
| 1755 | Minus | −319 | CGGGAGGCC | ERGTLAR | RSDNLAR | RSDHLRE | 0.006 |
| | | | (SEQ ID NO: 24) | (50) | (51) | (52) | |
| 1760 | Minus | −299 | ATGGCCGGG | RSDHLAR | DCRDLAR | RSDALTQ | 0.42 |
| | | | (SEQ ID NO: 25) | (53) | (54) | (55) | |
| 1757 | Minus | −290 | GGTGCGGAG | RSDNLTR | RSDDLNR | TSGHLVR | 0.05 |
| | | | (SEQ ID NO: 26) | (56) | (57) | (58) | |
| 1140 | Minus | −287 | AAGGGTGCG | RSDELTR | QSSHLAR | RSDNLTQ | 0.3 |
| | | | (SEQ ID NO: 27) | (59) | (60) | (61) | |
| 1764 | Plus | −286 | CAAGTGGGT | QSGHLAR | RSDALAR | QSGNLTE | 0.11 |
| | | | (SEQ ID NO: 28) | (62) | (63) | (64) | |
| 1772 | Plus | −277 | GTGGGTGAT | TTSNLAR | QSSHLAR | RSDALTR | 0.006 |
| | | | (SEQ ID NO: 29) | (65) | (66) | (67) | |
| 705 | Plus | −201 | GGGGCGGGG | RSDHLAR | RSDSLAR | RSDHLSR | 0.01 |
| | | | (SEQ ID NO: 30) | (68) | (69) | (70) | |
| 1750 | Plus | −147 | ATGGAGGGC | DRSHLTR | RSDNLAR | RSDALTE | 0.011 |
| | | | (SEQ ID NO: 31) | (71) | (72) | (73) | |
| 713 | Plus | −127 | GGTGAGGAG | RSDNLAR | RSDNLAR | MSHHLSR | 0.15 |
| | | | (SEQ ID NO: 32) | (74) | (75) | (76) | |
| 1796 | Plus | −124 | GAGGAGGCG | RSDDLTR | RSDNLAR | RSDNLAR | 0 |
| | | | (SEQ ID NO: 33) | (77) | (78) | (79) | |
| 386 | Plus | −111 | GGGGCGGAG | RSDNLTR | RSDELQR | RSDHLSR | 0.4 |
| | | | (SEQ ID NO: 34) | (80) | (81) | (82) | |
| 1610 | Plus | −86 | GCTGGGGCT | QSSDLRR | RSDHLTR | QSSDLRR | 0 |
| | | | (SEQ ID NO: 110) | (83) | (84) | (85) | |
| 1770 | Minus | −342 | CGGGGAGGA | QRAHLER | QSGHLQR | RSDHLRE | 0.01 |
| | | | (SEQ ID NO: 111) | (86) | (87) | (88) | |

¹An internal reference number;
²Target location is with respect to the translational initiation site (i.e., the A of the ATG codon);
³SEQ ID NO. given in parentheses;
⁴A K_{d} value of zero indicates that the binding constant was too low to be measured in the assay;
⁵recognition strand.

Three-finger ZFPs capable of binding to 9-10 bp target sites can be linked to form 6-finger proteins that bind to 18-20 bp target sites. See, for example, co-owned PCT WO 00/41566. ZFP DNA binding domains can also be linked to functional domains such as, for example, VP16, VP64 and p65 for transcriptional activation; and KRAB, MBD domains (e.g., MBD2B) or MeCP domains for transcriptional repression. Table 3 provides a listing of six-finger ZFPs with target sites in the human heparanase gene.

TABLE 3

Six-finger ZFPs with target sites in the human
heparanase gene

| | Target | | | |
|---|---|---|---|---|
| | loca- | Target | | Finger |
| SBS #¹ | tion² | strand | Target sequence | sequences^{3,4} |
| 5348 | −299 | minus | GGTGCGGAGATGGCCGGG | F1: RSAHLAR |
| | | | | (92) |
| | | | (SEQ ID NO: 89) | F2: DRSDLAR |
| | | | | (93) |
| | | | | F3: RSDALTQ |
| | | | | (94) |
| | | | | F4: RSANLAR |
| | | | | (95) |
| | | | | F5: RSDDLNR |
| | | | | (96 |
| | | | | F6: TSGHLVR |
| | | | | (97) |
| 5349 | −329 | minus | CGGGAGGCCAGGGGAGGA | F1: RSDNLTR |
| | | | | (98) |
| | | | G | F2: RSDNLAR |
| | | | | (99) |
| | | | (SEQ ID NO: 90) | F3: RSDHLSR |
| | | | | (100) |
| | | | | F4: ERGTLAR |
| | | | | (101) |
| | | | | F5: RSDNALR |
| | | | | (102) |
| | | | | F6: RSDHLRE |
| | | | | (103) |
| 5468 | −286 | plus | CAAGTGGGTGTGGGTGAT | F1: TTSNLAR |
| | | | | (104) |
| | | | (SEQ ID NO: 91) | F2: QSSHLAR |
| | | | | (105) |
| | | | | F3: RSDALTR |
| | | | | (106) |
| | | | | F4: QSGHLAR |
| | | | | (107) |
| | | | | F5: RSDALAR |
| | | | | (108) |
| | | | | F6: QSTNLKS |
| | | | | (109) |

¹An internal reference number.
²Target location is with respect to the translational initiation site (i.e., the A of the ATG codon)
³Sequence of amino acid residues −1 through +6, with respect to the first amino acid of the α-helix of the zinc finger.
⁴SEQ ID NO. given in parentheses

### Example 4

### Identification of Binding Sites for Transcription Factors in the Heparanase Gene

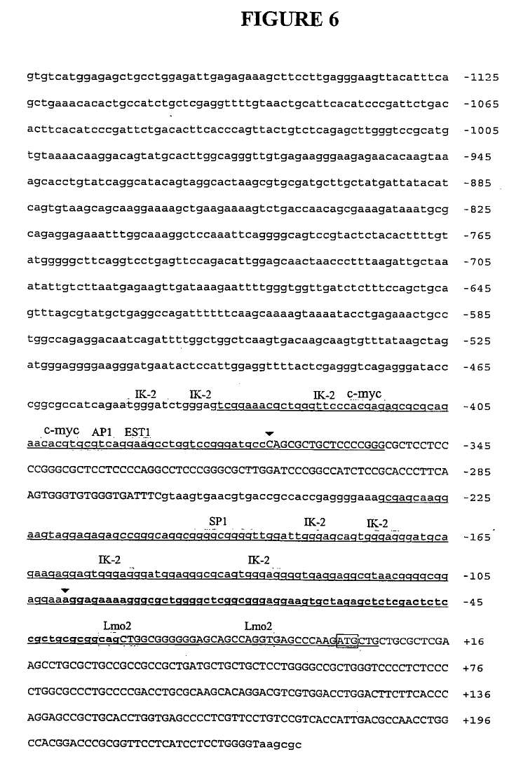

The nuclease hypersensitive regions of the heparanase sequence were analyzed, using the TRANSFAC program, to identify binding sites for transcription factors. See, for example, Wingender et al. (1997) Nucleic Acids Res. 25:265-268; Wingender et al. (2000) Nucleic Acids Res. 28:316-319; http://transfac.gbf.de/TRANSFAC/, accessed on Apr. 13, 2000. Results are presented in FIG. 6. Binding sites for SP1 (GGGGCGGGG, SEQ ID NO: 9), EST1 (AGGAAG, SEQ ID NO: 10) and AP1 (GCGTCA, SEQ ID NO: 11) were identified and their locations are indicated in FIG. 6. In addition, four E box sequences (CASSWG, SEQ ID NO: 12) were identified. Grutz et al. (1998) EMBO J. 17:45944605. In view of their flanking sequences and spacing, the two E boxes located at −34 bp and −14 bp (with respect to the translational startsite) in DHSS1 resemble Lmo2 binding sites. The Lmo2 gene encodes a nuclear LIM-domain protein, which is necessary for embryonic erythropoiesis and for adult haematopoiesis; and is activated in T-cell acute leukaemias by chromosomal translocations. The two E boxes located at −419 and −402 bp (with respect to the translational startsite) in the DHSS2 are more homologous to c-myc binding sites. The c-Myc protein binds to E boxes and transactivates genes. C-Myc induces neoplastic transformation and apoptosis and is involved in many human cancers. More strikingly, 7 potential IK-2 binding sites (TGGGAD, SEQ ID NO: 13) were located within the ˜460 bp DNA sequence covered by two DHS sites. The Ikaros gene encodes a zinc finger DNA-binding protein that is a potential regulator of lymphocyte commitment and differentiation. Alternatively spliced transcripts of the Ikaros gene encode at least 8 zinc finger proteins (IK-1 to IK-8) with distinct DNA binding capabilities and specificities. IK-2 protein can strongly stimulate transcription and is the predominant form of Ikaros in lymphocytes.

These transcription factor binding sites, which reside within regions of the sequence that have been identified as being hypersensitive to DNase, are likely to provide preferred binding sites, either for naturally-occurring transcription factors or designed zinc finger proteins, for exogenous regulation of the human heparanase gene. These sites are highlighted in FIG. 6.

### Example 5

### Determination of Heparanase mRNA Levels

To determine levels of expression of the human heparanase gene in various human tissues and cell lines, the TaqMan® real time RT-PCR technique was used. Accordingly, a probe/primer set for detection of endogenous heparanase was designed, and their sequences are presented in Table 4. The primers spans the 23-kb second intron of the heparanase gene, thereby generating a 161-nucleotide amplification product from mRNA, but not from genomic DNA and allowing quantitation of human heparanase mRNA from total RNA samples.

TABLE 4

Probe and Primers for Real-Time PCR

| | | SEQ ID | Loca- |
|---|---|---|---|
| Strand | Sequence (5′→3′) | NO | tion |
| Forward | CAAGCACAGGACGTCGTGGA | 14 | +99 |
| Probe | CTCGTTCCTGTCCGTCACCATTGAC | 15 | +162 |
| Reverse | AGCCTCTGGCCAAGGTACGA | 16 | +259 |

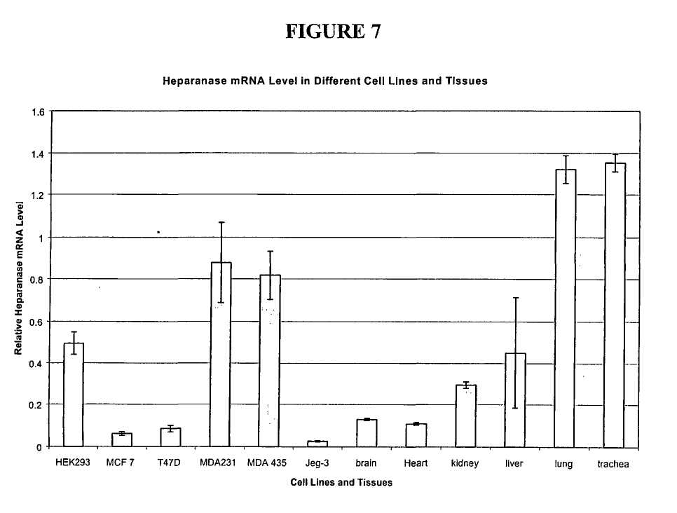

Total RNA was either isolated from cultured cells using a RNeasy miniprep kit (Qiagen, Valencia, Calif.) or purchased from Clontech (Palo Alto, Calif.). A relative quantitation with standard curve method (Applied BioSystems, Foster City, Calif., TaqMan User Bulletin #2) was used to quantitate heparanase mRNA levels in each RNA preparation. Human GAPDH RNA or 18S ribosomal RNA was used to normalize the total RNA input for each reaction. Results for several different cell lines and human tissues are shown in FIG. 7. Under normal culture conditions, MDA435 and MDA231 (two highly invasive mammary tumor cell lines) contained ˜20 fold higher levels of heparanase mRNA than the less invasive mammary tumor lines MCF7 and T47D. Jeg-3 cells (a choriocarcinoma cell line) expressed the lowest level of heparanase among all cells and tissue tested, about 30-fold less than MDA435. HEK 293 cells (a transformed human embryonic kidney line) expressed intermediate levels of heparanase mRNA. Among the tissues tested, lung and trachea showed highest expression levels, while brain and heart expressed heparanase mRNA at about a 10-fold lower level. The expression results correlate with the degree of invasiveness of the tumor from which each of these cell lines was derived, in that cell lines derived from more invasive tumors had higher heparanase mRNA levels.

### Example 6

### Invasiveness Assay

Heparanase expression is closely linked to cell migration and invasiveness. Accordingly, inhibition of heparanase expression in a cell, using compositions and methods disclosed herein, is likely to be accompanied by reduced invasiveness of the cell. Cellular invasivenesss is assessed using a Boyden Chamber assay. Biocoat Matrigel invasion chambers for use in this assay are available from Becton Dickinson Labware (Bedford, Mass.). These units are separated into upper and lower chambers by 8 um poycarbonate membranes. The membranes are coated with Matrigel from Engelbreth-HolmSwarm Murine Sarcoma.

Cells are seeded in the upper chamber of the unit, in medium containing 0.1% fetal bovine serum. The medium in the lower chamber of the unit contains 10% fetal bovine serum. Cells are incubated in the unit at 37° C. for a given time period (e.g., 4 hours), then the membranes are fixed and stained with trypan blue. After removal of cells that have not migrated (e.g., by wiping the appropriate surface of the membrane with a cotton swab), cells that have migrated through the membrane to its lower surface, are counted under a microscope.

Cells that have been treated with the disclosed compositions (e.g., fusion proteins targeted to a heparanase regulatory sequence) are compared to untreated cells in this assay. Treatment of cells with fusion proteins comprising a DNA binding domain targeted to a heparanase sequence and a repressive functional domain will result in reduced invasiveness of the treated cells, compared to untreated control cells.

### Example 7

### Regulation of the Human Heparanase Gene by ZFPs

A number of the ZFP DNA-binding domains listed in Tables 2 and 3 were fused to functional domains and tested for their ability to regulate expression of the human heparanase gene in living cells. The functional domains used in these experiments were the VP16 and p65 activation domains, and the cells used were the PC-3 human prostate cancer cell line and the HEK 293 human embryonic kidney cell line. Nucleic acid vectors encoding fusion molecules comprising a given ZFP DNA-binding domain, a VP16 or p65 activation domain, a nuclear localization signal and an epitope tag were constructed as described, for example in co-owned WO 00/41566 and WO 00/42219, Zhang et al. (2000) J. Biol. Chem. 275:33,850-33,860 and Liu et al., (2001) J. Biol. Chem. 276:11,323-11,334, the disclosures of which are hereby incorporated by reference in their entireties. Cells were cultured and transfected as described, for example in co-owned WO 00/41566 and WO 00/42219, Zhang et al. (2000) J. Biol. Chem. 275:33,850-33,860 and Liu et al. (2001) J. Biol. Chem. 276:11,323-11,334, the disclosures of which are hereby incorporated by reference in their entireties. Heparanase mRNA level were measured as described in Example 5.

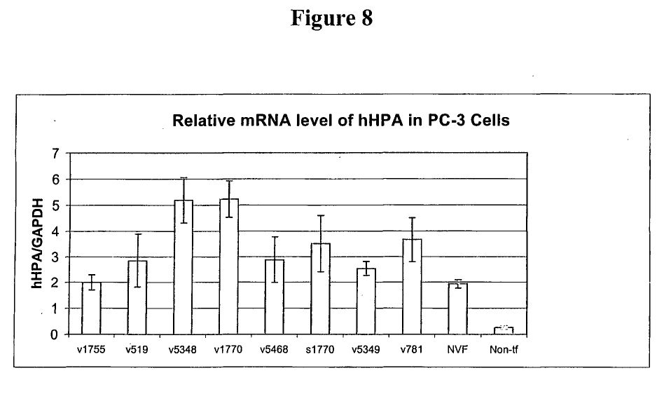

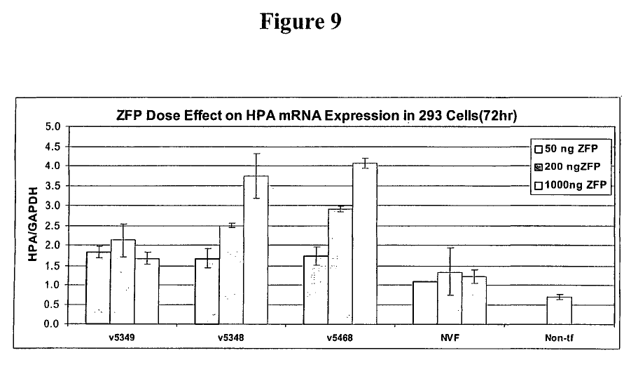

The results of these analyses, shown in FIGS. 8 and 9, indicate that, of the eight ZFP-activation domain fusions tested, 6 were able to increase heparanase mRNA levels in transfected cells.

### Example 8

### Identification of Human Heparanase Gene Regulatory Sequences

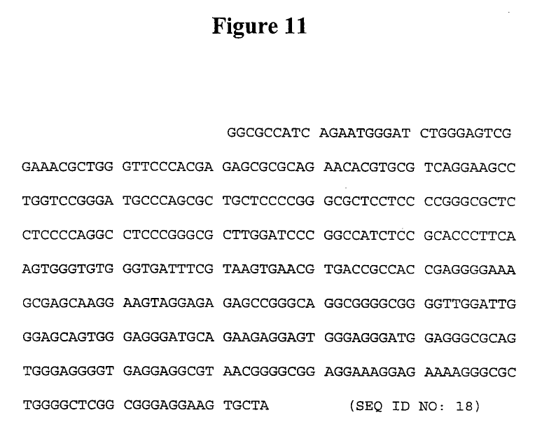

The locations of the target sites of the ZFP fusions that were tested in Example 7 were determined. These are shown in FIG. 10. The target sites fall within both hypersensitive regions and also lie within the region between the two hypersensitive sites identified by DNase I digestion. As noted supra, regulatory regions may extend beyond the boundaries of a region of cellular chromatin that is preferentially susceptible to nuclease action. Accordingly, it is determined that the human heparanase regulatory region includes both the regions that are hypersensitive to DNase I and the region therebetween, in which lie several target sites for ZFPs capable of modulating heparanase gene expression. The sequence of this heparanase regulatory region, which lies approximately between a Ban I site and a Sac I site, is presented in FIG. 11 (SEQ ID NO.: 18).

What is claimed is:

1. An isolated polynucleotide comprising a heparanese sequence having X contiguous nucleotides, wherein (i) the X contiguous nucleotides have at least about 80% identity to Y contiguous nucleotides derived from SEQ ID NO:2, (ii) X equals Y, and (iii) X is greater than or equal to 50.

2. The isolated polynucleotide of claim 1, wherein X is between about 50 and 650, including all integer values between 50 and 650.

3. The isolated polynucleotide of claim 1, wherein X is greater than or equal to 650.

4. An isolated polynucleotide comprising SEQ ID NO:2.

5. An isolated polynucleotide comprising a heparanese sequence having X contiguous nucleotides, wherein (i) the X contiguous nucleotides have at least about 80% identity to Y contiguous nucleotides derived from SEQ ID NO:3, (ii) X equals Y, and (iii) X is greater than or equal to 50.

6. The isolated polynucleotide of claim 5, wherein X is between about 50 and 650, including all integer values between 50 and 650.

7. The isolated polynucleotide of claim 1, wherein X is greater than or equal to 650.

8. An isolated polynucleotide comprising SEQ ID NO:3.

9. An expression vector comprising the isolated polynucleotide according to claim 1.

10. An expression vector comprising the isolated polynucleotide according to claim 5.

11. A host cell comprising the isolated polynucleotide according to claim 1.

12. A host cell comprising the isolated polynucleotide according to claim 5.

13. A fusion polypeptide comprising
(a) a DNA binding domain targeted to a region of the isolated polynucleotide of claim 1 or claim 5; and
(b) a transcriptional regulatory domain or functional fragment thereof.

14. The fusion polypeptide of claim 13, wherein the DNA binding domain is a zinc finger DNA binding domain.

15. The fusion polypeptide of claim 14, wherein the targeted region is at least 9 nucleotides in length.

16. The fusion polypeptide of claim 13, wherein the transcriptional regulatory domain comprises a repression domain.

17. The fusion polypeptide of claim 16, wherein the repression domain is selected from the group consisting of (a) KRAB; (b) MBD2B;
(c) v-erbA and (d) functional fragments of (a), (b) or (c).

18. The fusion polypeptide of claim 13, wherein the transcriptional regulatory domain comprises an activation domain.

19. The fusion polypeptide of claim 18, wherein the activation domain is selected from the group consisting of (a) VP 16; (b) p65 and (c) functional fragments of (a) or (b).

20. A polynucleotide encoding the fusion polypeptide of claim 13.

21. A cell comprising the polynucleotide of claim 20.

22. A cell comprising the fusion polypeptide of claim 13.

23. A method of modulating expression of a heparanase gene, the method comprising the step of contacting a region of SEQ ID NO:2 or SEQ ID NO:3 with a molecule that binds to a binding site in the region.

24. The method of claim 23, wherein the molecule is a fusion molecule comprising a DNA binding domain and a transcriptional regulatory domain or functional fragments thereof.

25. The method of claim 23, wherein the molecule is an endogenous transcriptional regulatory factor.

26. The method of claim 23, wherein the region is in the isolated polynucleotide of claim 1.

27. The method of claim 23, wherein the region is in the isolated polynucleotide of claim 5.

28. The method of claim 23, wherein the region is at least 9 nucleotides in length.

29. The method of claim 23, wherein the modulation of the heparanase gene comprises repression of heparanase.

30. The method of claim 29, wherein the transcriptional regulatory domain comprises a repression domain.

31. The method of claim 30, wherein the repression domain is selected from the group consisting of (a) KRAB; (b) MBD2B; (c) v-erbA and (d) functional fragments of (a), (b) or (c).

32. The method of claim 23, wherein the modulation of the heparanase gene comprises activation of heparanase.

33. The method of claim 32, wherein the transcriptional regulatory domain comprises an activation domain.

34. The method of claim 33, wherein the activation domain is selected from the group consisting of (a) VP16; (b) p65; and (c) functional fragments of (a) or (b).

35. The method of claim 23, wherein the heparanase gene is in a plant cell.

36. The method of claim 23, wherein the heparanase gene is in an animal cell.

37. The method of claim 36, wherein the animal cell is a human cell.

38. A recombinant expression construct effective in directing the transcription of a selected coding sequence, said expression construct comprising:
(a) a coding sequence; and
(b) control elements that are operably linked to said coding sequence, wherein said control elements comprise a polynucleotide derived from a polynucleotide according to claim 1 or claim 5 or a functional fragment thereof, and wherein said coding sequence can be transcribed and translated in a host cell.

39. A host cell transformed with the recombinant expression construct of claim 38.

40. A method of modulating expression of a target coding sequence in a host cell comprising the step of contacting the host cell with an expression construct according to claim 38, wherein the expression construct comprises the target coding sequence.

41. An isolated polynucleotide comprising a heparanese sequence having X contiguous nucleotides, wherein (i) the X contiguous nucleotides have at least about 80% identity to Y contiguous nucleotides derived from SEQ ID NO:18, (ii) X equals Y, and (iii) X is greater than or equal to 50.

42. The isolated polynucleotide of claim 41, wherein X is between about 50 and 650, including all integer values between 50 and 650.

43. The isolated polynucleotide of claim 41, wherein X is greater than or equal to 650.

44. An isolated polynucleotide comprising SEQ ID NO: 18.

45. An expression vector comprising the isolated polynucleotide according to claim 41.

46. A host cell comprising the isolated polynucleotide according to claim 41.

47. A fusion polypeptide comprising
(c) a DNA binding domain targeted to a region of the isolated polynucleotide of claim 41; and
(d) a transcriptional regulatory domain or functional fragment thereof.

48. The fusion polypeptide of claim 47, wherein the DNA binding domain is a zinc finger DNA binding domain.

49. The fusion polypeptide of claim 48, wherein the targeted region is at least 9 nucleotides in length.

50. The fusion polypeptide of claim 47, wherein the transcriptional regulatory domain comprises a repression domain.

51. The fusion polypeptide of claim 50, wherein the repression domain is selected from the group consisting of (a) KRAB; (b) MBD2B; (c) v-erbA and (d) functional fragments of (a), (b) or (c).

52. The fusion polypeptide of claim 47, wherein the transcriptional regulatory domain comprises an activation domain.

53. The fusion polypeptide of claim 52, wherein the activation domain is selected from the group consisting of (a) VP 16; (b) p65 and (c) functional fragments of (a) or (b).

54. A polynucleotide encoding the fusion polypeptide of claim 47.

55. A cell comprising the polynucleotide of claim 54.

56. A cell comprising the fusion polypeptide of claim 47.

57. A method of modulating expression of a heparanase gene, the method comprising the step of contacting a region of SEQ ID NO: 18, or a functional fragment thereof, with a molecule that binds to a binding site in the region.

58. The method of claim 57, wherein the molecule is a fusion molecule comprising a DNA binding domain and a transcriptional regulatory domain or functional fragments thereof.

59. The method of claim 57, wherein the molecule is an endogenous transcriptional regulatory factor.

60. The method of claim 57, wherein the region is at least 9 nucleotides in length.

61. The method of claim 57, wherein the modulation of the heparanase gene comprises repression of heparanase gene expression.

62. The method of claim 61, wherein the transcriptional regulatory domain comprises a repression domain.

63. The method of claim 62, wherein the repression domain is selected from the group consisting of (a) KRAB; (b) MBD2B; (c) v-erbA and (d) functional fragments of (a), (b) or (c).

64. The method of claim 57, wherein the modulation of the heparanase gene comprises activation of heparanase gene expression.

65. The method of claim 64, wherein the transcriptional regulatory domain comprises an activation domain.

66. The method of claim 65, wherein the activation domain is selected from the group consisting of (a) VP16; (b) p65; and (c) functional fragments of (a) or (b).

67. The method of claim 57, wherein the heparanase gene is in an animal cell.

68. The method of claim 67, wherein the cell is a human cell.

69. A recombinant expression construct effective in directing the transcription of a selected coding sequence, said expression construct comprising:
(b) a coding sequence; and
(b) control elements that are operably linked to said coding sequence, wherein said control elements comprise a polynucleotide derived from a polynucleotide according to claim 41 or a functional fragment thereof, and wherein said coding sequence can be transcribed and translated in a host cell.

70. A host cell transformed with the recombinant expression construct of claim 69.

71. A method of modulating expression of a target coding sequence in a host cell comprising the step of contacting the host cell with an expression construct according to claim 69, wherein the expression construct comprises the target coding sequence.
